(19) United States
(12) Patent Application Publication
(10) Pub. No.: US 2008/0112909 A1
(43) Pub. Date: May 15, 2008

(54) COMPOSITIONS FOR PROVIDING COLOR TO ANIMATE OBJECTS AND RELATED METHODS

(75) Inventors: Dennis L. Faler, North Huntingdon, PA (US); Gregory J. McCollum, Gibsonia, PA (US); Calum H. Munro, Wexford, PA (US); W. David Polk, Pittsburgh, PA (US)

Correspondence Address: PPG INDUSTRIES INC;INTELLECTUAL PROPERTY DEPT, ONE PPG PLACE, PITTSBURGH, PA, 15272, US

(73) Assignee: PPG INDUSTRIES OHIO, INC., Cleveland, OH (US)

(21) Appl. No.: 12/017,541

(22) Filed: Jan. 22, 2008

Related U.S. Application Data
(63) Continuation-in-part of application No. 11/337,062, filed on Jan. 20, 2006.
Application No. 11/337,062 is a continuation-in-part of application No. 10/876,031, filed on Jun. 24, 2004.
(60) Provisional application No. 60/886,142, filed on Jan. 23, 2007.
(60) Provisional application No. 60/482,167, filed on Jun. 24, 2003.

(51) Int. Cl. A61K 8/18

(52) U.S. Cl. 424/61; 424/63; 424/401; 977/926

(57) ABSTRACT

Methods for providing color to animate objects, i.e., living things, as well as compositions, such as personal care compositions and agrochemical compositions, suitable for application to such animate objects. The compositions include colorants that include a radiation diffraction material comprising an ordered periodic array of particles held in a matrix, and/or a dispersion of polymer-enclosed nanoparticles.

### CROSS-REFERENCE TO RELATED APPLICATIONS

This application claims the benefit of U.S. Provisional Patent Application Ser. No. 60/886,142, filed Jan. 23, 2007, incorporated herein by reference. This application is a continuation-in-part of U.S. patent application Ser. No. 11/337,062, entitled “Aqueous Dispersions of Polymer-Enclosed Particles, Related Coating Compositions and Coated Substrates, filed Jan. 20, 2006, which is a continuation-in-part of U.S. patent application Ser. No. 10/876,031, entitled, “Aqueous Dispersions of Microparticles Having a Nanoparticulate Phase and Coating Compositions Containing The Same”, filed Jun. 24, 2004, which claims the benefit of U.S. Provisional Application Ser. No. 60/482,167 filed Jun. 24, 2003, each of which are incorporated herein by reference.

### FIELD OF THE INVENTION

The present invention relates to compositions for providing color to animate objects, i.e., living things, such as personal care compositions and agrochemical compositions, suitable for application to such animate objects.

### BACKGROUND INFORMATION

Animate objects, such as (1) plant life, including turf, trees, shrubs, crops, seeds, and the like, and (2) animals often have a composition applied thereto for cosmetic, pharmaceutical, nutritional, herbicidal, decorative, protective, and/or other purposes. For example, in the case of animals and human beings, compositions are often applied to the body, face, fur, hair, nails, and/or teeth. Agrochemical compositions are often applied to seeds and plant life to aid or contribute to the germination of a seed or growth of a seedling.

In many cases, these compositions include colorants. For example, personal care compositions, including cosmetic compositions, such as makeup compositions, including free or compacted powders, foundations, face powders, eyeshadows, lipsticks, products for concealing rings under the eyes, blushers, mascaras, eyeliners, lip pencils, eyeliner pencils, nail varnishes and other products for making up the body often include an appropriate vehicle and a coloring agent intended to confer a certain color on these compositions before and/or after their application to the skin, lips, hair, and/or other body growths.

These coloring agents are often dyes, inorganic or organic pigments, and/or pearlescent pigments. In the red pigments range, for example, cosmetic scientists have available pigments of inorganic origin, such as red iron oxides or mixtures of brown-yellow iron oxides, and pigments of organic origin. Historically, inorganic pigments, in particular inorganic oxides, have had the advantage of being very stable but have the disadvantage of giving rather drab and pale colors. Organic dyes, historically, have had the advantage of conferring vivid colors on the compositions but many are (i) unstable with respect to light, temperature or pH, and/or (ii) subject to migration/bleeding. Pearlescent pigments, for their part, have made it possible to often obtain varied but never intense colors with effects which are iridescent but which are generally fairly weak.

As a result, it would be desirable to provide compositions of matter, such as personal care compositions and agrochemical compositions, suitable for application to animate objects, which contain improved colorants.

### SUMMARY OF THE INVENTION

In certain respects, the present invention is directed to personal care compositions comprising a colorant, wherein the colorant comprises: (a) a radiation diffraction material comprising an ordered periodic array of particles held in a matrix, (b) a dispersion of polymer-enclosed nanoparticles, or (c) a mixture thereof.

In other respects, the present invention is directed to agrochemical compositions comprising (a) an agricultural active ingredient; and (b) a colorant, wherein the colorant comprises: (i) a radiation diffraction material comprising an ordered periodic array of particles held in a matrix, (ii) a dispersion of polymer-enclosed nanoparticles, or (iii) a mixture thereof.

In certain respects, the present invention is directed to methods for coloring an animate object comprising applying an effective amount of a composition comprising a colorant, wherein the colorant comprises (i) a radiation diffraction material comprising an ordered periodic array of particles held in a matrix, (ii) a dispersion of polymer-enclosed nanoparticles, or (iii) a mixture thereof.

In other respects, the present invention is directed to methods for treating a keratinous substrate, comprising applying to at least a portion of the substrate a treatment composition comprising a colorant, wherein the colorant comprises (i) a radiation diffraction material comprising an ordered periodic array of particles held in a matrix, (ii) a dispersion of polymer-enclosed nanoparticles, or (iii) a mixture thereof.

In still other respects, the present invention is directed to methods for protecting a keratinous substrate from environmental damage, comprising applying to at least a portion of the substrate a composition comprising a colorant, wherein the colorant comprises (i) a radiation diffraction material comprising an ordered periodic array of particles held in a matrix, (ii) a dispersion of polymer-enclosed nanoparticles, or (iii) a mixture thereof.

The present invention is also directed to non-human multi-cellular organisms that are at least partially coated with a composition comprising a colorant comprising: (a) a radiation diffraction material comprising an ordered periodic array of particles held in a matrix, (b) a dispersion of polymer-enclosed nanoparticles, or (c) a mixture thereof.

### DETAILED DESCRIPTION OF THE INVENTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances.

As previously indicated, the present invention, in certain embodiments, is directed to personal care compositions. As used herein, the term “personal care composition” refers to a product suitable for application to a keratinous substrate, i.e., the skin, hair (including eyelashes and eyebrows), nails, oral cavity and related membranes, for the purpose of improving, cleaning, beautifying, treating, and/or caring for these surfaces and membranes. As a result, personal care compositions include, but are not necessarily limited to, (1) cosmetic compositions and (2) pharmaceutical or neutraceutical compositions, such as drugs. Moreover, the term “personal care composition”, as used herein, is intended to cover such compositions intended for use on humans as well as animals other than humans.

As used herein, the term “cosmetic composition” refers to compositions that are suitable for introduction into, or application on, the human body or any part thereof, by, for example, rubbing, brushing, pouring, sprinkling, or spraying, for the purpose of cleansing, beautifying, promoting attractiveness, or altering the appearance thereof. Such compositions provide a cosmetic effect and are not regulated as pharmaceuticals. In some situations, however, cosmetic compositions are incorporated in pharmaceutical compositions to provide cosmetic effects (e.g., products that treat skin or hair diseases, but also contain a cosmetic composition).

As used herein, the term “pharmaceutical composition” refers to compositions, such as drugs, that provide a medical effect, rather than solely cosmetic benefits. As used herein, the term “neutraceutical composition” refers to compositions that provide both nutritional benefits as well as the benefits of a pharmaceutical composition.

As used herein, the term “cosmetic effect” refers to a cosmetic benefit resulting from the administration of a personal care composition. Cosmetic benefits include, but are not limited to, maintenance, improvement and/or change in the condition of, for example, cleanliness, color, beauty, or other appearance of, for example, skin, hair, nails, and the oral cavity. As used herein, the term “medical effect” refers to a benefit on the structure or function of the body of a human being or animal, including, for example, the diagnosis, cure, mitigation, treatment, or prevention of a disease.

The personal care compositions of the present invention may be embodied in any of a variety of cosmetic and/or pharmaceutical compositions. Non-limiting examples of which include skin care compositions, hair care compositions, nail care compositions, makeup compositions and oral hygiene compositions.

As used herein, the term “skin care composition” refers to personal care compositions that are suitable for application to the skin to provide, for example, a cosmetic effect and/or a medical effect, such as, protection from ultraviolet rays, wrinkle minimizing, wrinkle removal, skin tanning, skin softening, skin smoothing, depilation, cleansing, etc. In certain embodiments, the present invention provides skin care compositions that improve or change skin tone.

As used herein, the term “hair care composition” refers to personal care compositions that are suitable for application to hair to provide, for example, a cosmetic effect and/or a medical effect, such as thickening, thinning, coloring, decoloring, cleansing, conditioning, softening, shaping, and the like.

As used herein the term “nail care composition” refers to personal care compositions that are suitable for application to the nails to provide, for example, a cosmetic effect and/or a medical effect, such as harder and stronger nails, a change in nail color, and the like.

As used herein, the term “makeup composition” refer to personal care compositions that are suitable for beautifying, caring for, maintaining, or augmenting the appearance of a human or other animal. “Makeup compositions” include, but are not limited to, mascaras, lipsticks, lip-liners, tattoos, including temporary tattoos, eye shadows, eye-liners, rouges, face powders, foundations, blushes, and nail polish.

As used herein, the term “oral hygiene composition” refers to personal care compositions that are suitable for application to the oral cavity to provide, for example, a cosmetic effect and/or a medical effect, such as cleansing, disinfecting, and the like. As a result, oral hygiene compositions include, for example, mouthwashes and rinses, toothpaste, gels, powders, gums, mouth sprays and lozenges.

As previously indicated, the personal care compositions of the present invention comprise a colorant. As used herein, the term “colorant” refers to any substance that imparts color and/or other opacity and/or other visual effect to the composition in which it is present. In the methods and compositions of the present invention, a single colorant or a mixture of two or more colorants may be used.

In certain embodiments, the colorant present in the composition of the present invention comprises polymer-enclosed color-imparting particles. As used herein, the term “polymer-enclosed particles” refers to particles that are at least partially enclosed by, i.e., confined within, a polymer to an extent sufficient to separate particles from each other within the resulting coating composition, such that significant agglomeration of the particles is prevented. It will be appreciated, of course, that a composition of the present invention that comprises such “polymer-enclosed particles” may also include particles that are not polymer-enclosed particles. As used herein, the term “color-imparting particle” refers to a particle that significantly absorbs some wavelengths of visible light, that is, wavelengths ranging from 400 to 700 nanometers, more than it absorbs other wavelengths in the visible region.

In certain embodiments, the particles that are enclosed by a polymer in the compositions of the present invention comprise nanoparticles. As used herein, the term “nanoparticle” refers to a particle that has a particle size of less than 1 micron. In certain embodiments, the nanoparticles used in the present invention have an average particle size of 300 nanometers or less, such as 200 nanometers or less, or, in some cases, 100 nanometers or less.

For purposes of the present invention, average particle size can be measured according to known laser scattering techniques. For example, average particle size can be determined using a Horiba Model LA 900 laser diffraction particle size instrument, which uses a helium-neon laser with a wave length of 633 nanometers to measure the size of the particles and assumes the particle has a spherical shape, i.e., the “particle size” refers to the smallest sphere that will completely enclose the particle. Average particle size can also be determined by visually examining an electron micrograph of a transmission electron microscopy (“TEM”) image of a representative sample of the particles, measuring the diameter of the particles in the image, and calculating the average primary particle size of the measured particles based on magnification of the TEM image. One of ordinary skill in the art will understand how to prepare such a TEM image and determine the primary particle size based on the magnification. The primary particle size of a particle refers to the smallest diameter sphere that will completely enclose the particle. As used herein, the term “primary particle size” refers to the size of an individual particle as opposed to an agglomeration of two or more individual particles.

The shape (or morphology) of the polymer-enclosed color-imparting particles can vary. For example, generally spherical morphologies (such as solid beads, microbeads, or hollow spheres), can be used, as well as particles that are cubic, platy, or acicular (elongated or fibrous). Additionally, the particles can have an internal structure that is hollow, porous or void free, or a combination of any of the foregoing, e.g., a hollow center with porous or solid walls. For more information on suitable particle characteristics see H. Katz et al. (Ed.), Handbook of Fillers and Plastics (1987) at pages 9-10.

Depending on the desired properties and characteristics of the resultant composition, mixtures of one or more polymer-enclosed color-imparting particles having different average particle sizes can be employed.

The polymer-enclosed color-imparting particles, such as nanoparticles, can be formed from any of a variety of materials, such as polymeric and/or non-polymeric inorganic materials, polymeric and/or non-polymeric organic materials, composite materials, as well as mixtures of any of the foregoing. As used herein, “formed from” denotes open, e.g., “comprising,” claim language. As such, it is intended that a composition or substance “formed from” a list of recited components be a composition comprising at least these recited components, and can further comprise other, non-recited components, during the composition's formation. Additionally, as used herein, the term “polymer” is meant to encompass oligomers, and includes without limitation both homopolymers and copolymers.

As used herein, the term “polymeric inorganic material” means a polymeric material having a backbone repeat unit based on an element or elements other than carbon. Moreover, as used herein, the term “polymeric organic materials” means synthetic polymeric materials, semi-synthetic polymeric materials and natural polymeric materials, all of which have a backbone repeat unit based on carbon.

The term “organic material,” as used herein, means carbon containing compounds wherein the carbon is typically bonded to itself and to hydrogen, and often to other elements as well, and excludes binary compounds such as the carbon oxides, the carbides, carbon disulfide, etc.; such ternary compounds as the metallic cyanides, metallic carbonyls, phosgene, carbonyl sulfide, etc.; and carbon-containing ionic compounds such as metallic carbonates, for example calcium carbonate and sodium carbonate.

As used herein, the term “inorganic material” means any material that is not an organic material.

As used herein, the term “composite material” means a combination of two or more differing materials. The particles formed from composite materials generally have a hardness at their surface that is different from the hardness of the internal portions of the particle beneath its surface. More specifically, the surface of the particle can be modified in any manner well known in the art, including, but not limited to, chemically or physically changing its surface characteristics using techniques known in the art.

For example, a particle can be formed from a primary material that is coated, clad or encapsulated with one or more secondary materials to form a composite particle that has a softer surface. In certain embodiments, particles formed from composite materials can be formed from a primary material that is coated, clad or encapsulated with a different form of the primary material. For more information on particles useful in the present invention, see G. Wypych, Handbook of Fillers, 2nd Ed. (1999) at pages 15-202.

As aforementioned, the particles useful in the present invention can include any inorganic materials known in the art. Suitable particles can be formed from ceramic materials, metallic materials, and mixtures of any of the foregoing. Non-limiting examples of such ceramic materials can comprise metal oxides, mixed metal oxides, metal nitrides, metal carbides, metal sulfides, metal silicates, metal borides, metal carbonates, and mixtures of any of the foregoing. A specific, non-limiting example of a metal nitride is boron nitride; a specific, non-limiting example of a metal oxide is zinc oxide; non-limiting examples of suitable mixed metal oxides are aluminum silicates and magnesium silicates; non-limiting examples of suitable metal sulfides are molybdenum disulfide, tantalum disulfide, tungsten disulfide, and zinc sulfide; non-limiting examples of metal silicates are aluminum silicates and magnesium silicates, such as vermiculite.

In certain embodiments of the present invention, the particles comprise inorganic materials selected from aluminum, barium, bismuth, boron, cadmium, calcium, cerium, cobalt, copper, iron, lanthanum, magnesium, manganese, molybdenum, nitrogen, oxygen, phosphorus, selenium, silicon, silver, sulfur, tin, titanium, tungsten, vanadium, yttrium, zinc, and zirconium, including oxides thereof, nitrides thereof, phosphides thereof, phosphates thereof, selenides thereof, sulfides thereof, sulfates thereof, and mixtures thereof. Suitable non-limiting examples of the foregoing inorganic particles are alumina, silica, titania, ceria, zirconia, bismuth oxide, magnesium oxide, iron oxide, aluminum silicate, boron carbide, nitrogen doped titania, and cadmium selenide.

The particles can comprise, for example, a core of essentially a single inorganic oxide, such as silica in colloidal, fumed, or amorphous form, alumina or colloidal alumina, titanium dioxide, iron oxide, cesium oxide, yttrium oxide, colloidal yttria, zirconia, e.g., colloidal or amorphous zirconia, and mixtures of any of the foregoing; or an inorganic oxide of one type upon which is deposited an organic oxide of another type.

Non-polymeric, inorganic materials useful in forming the particles used in the present invention can comprise inorganic materials selected from graphite, metals, oxides, carbides, nitrides, borides, sulfides, silicates, carbonates, sulfates, and hydroxides. A non-limiting example of a useful inorganic oxide is zinc oxide. Non-limiting examples of suitable inorganic sulfides include molybdenum disulfide, tantalum disulfide, tungsten disulfide, and zinc sulfide. Non-limiting examples of useful inorganic silicates include aluminum silicates and magnesium silicates, such as vermiculite. Non-limiting examples of suitable metals include molybdenum, platinum, palladium, nickel, aluminum, copper, gold, iron, silver, alloys, and mixtures of any of the foregoing.

In certain embodiments, the particles are selected from fumed silica, amorphous silica, colloidal silica, alumina, colloidal alumina, titanium dioxide, iron oxide, cesium oxide, yttrium oxide, colloidal yttria, zirconia, colloidal zirconia, and mixtures of any of the foregoing. In certain embodiments, the particles comprise colloidal silica. As disclosed above, these materials can be surface treated or untreated. Other useful particles include surface-modified silicas, such as are described in U.S. Pat. No. 5,853,809 at column 6, line 51 to column 8, line 43, incorporated herein by reference.

As another alternative, a particle can be formed from a primary material that is coated, clad or encapsulated with one or more secondary materials to form a composite material that has a harder surface. Alternatively, a particle can be formed from a primary material that is coated, clad or encapsulated with a differing form of the primary material to form a composite material that has a harder surface.

In one example, and without limiting the present invention, an inorganic particle formed from an inorganic material, such as silicon carbide or aluminum nitride, can be provided with a silica, carbonate or nanoclay coating to form a useful composite particle. In another non-limiting example, a silane coupling agent with alkyl side chains can interact with the surface of an inorganic particle formed from an inorganic oxide to provide a useful composite particle having a “softer” surface. Other examples include cladding, encapsulating or coating particles formed from non-polymeric or polymeric materials with differing non-polymeric or polymeric materials. A specific non-limiting example of such composite particles is DUALITE™, which is a synthetic polymeric particle coated with calcium carbonate that is commercially available from Pierce and Stevens Corporation of Buffalo, N.Y.

In certain embodiments, the particles used in the present invention have a lamellar structure. Particles having a lamellar structure are composed of sheets or plates of atoms in hexagonal array, with strong bonding within the sheet and weak van der Waals bonding between sheets, providing low shear strength between sheets. A non-limiting example of a lamellar structure is a hexagonal crystal structure. Inorganic solid particles having a lamellar fullerene (i.e., buckyball) structure are also useful in the present invention.

Non-limiting examples of suitable materials having a lamellar structure include boron nitride, graphite, metal dichalcogenides, mica, talc, gypsum, kaolinite, calcite, cadmium iodide, silver sulfide and mixtures thereof. Suitable metal dichalcogenides include molybdenum disulfide, molybdenum diselenide, tantalum disulfide, tantalum diselenide, tungsten disulfide, tungsten diselenide and mixtures thereof.

The particles can be formed from non-polymeric, organic materials. Non-limiting examples of non-polymeric, organic materials useful in the present invention include, but are not limited to, stearates (such as zinc stearate and aluminum stearate), diamond, carbon black and stearamide.

The particles used in the present invention can be formed from inorganic polymeric materials. Non-limiting examples of useful inorganic polymeric materials include polyphosphazenes, polysilanes, polysiloxanes, polygermanes, polymeric sulfur, polymeric selenium, silicones and mixtures of any of the foregoing. A specific, non-limiting example of a particle formed from an inorganic polymeric material suitable for use in the present invention is Tospearl, which is a particle formed from cross-linked siloxanes and is commercially available from Toshiba Silicones Company, Ltd. of Japan.

The particles can be formed from synthetic, organic polymeric materials. Non-limiting examples of suitable organic polymeric materials include, but are not limited to, thermoset materials and thermoplastic materials. Non-limiting examples of suitable thermoplastic materials include thermoplastic polyesters, such as polyethylene terephthalate, polybutylene terephthalate and polyethylene naphthalate, polycarbonates, polyolefins, such as polyethylene, polypropylene and polyisobutene, acrylic polymers, such as copolymers of styrene and an acrylic acid monomer and polymers containing methacrylate, polyamides, thermoplastic polyurethanes, vinyl polymers, and mixtures of any of the foregoing.

Non-limiting examples of suitable thermoset materials include thermoset polyesters, vinyl esters, epoxy materials, phenolics, aminoplasts, thermoset polyurethanes and mixtures of any of the foregoing. A specific, non-limiting example of a synthetic polymeric particle formed from an epoxy material is an epoxy microgel particle.

The particles can also be hollow particles formed from materials selected from polymeric and non-polymeric inorganic materials, polymeric and non-polymeric organic materials, composite materials and mixtures of any of the foregoing. Non-limiting examples of suitable materials from which the hollow particles can be formed are described above.

In certain embodiments, the particles used in the present invention comprise an organic pigment, for example, azo compounds (monoazo, di-azo, β-Naphthol,Naphthol AS salt type azo pigment lakes, benzimidazolone, di-azo condensation, isoindolinone, isoindoline), and polycyclic (phthalocyanine, quinacridone, perylene, perinone, diketopyrrolo pyrrole, thioindigo, anthraquinone, indanthrone, anthrapyrimidine, flavanthrone, pyranthrone, anthanthrone, dioxazine, triarylcarbonium, quinophthalone) pigments, and mixtures thereof. In certain embodiments, the organic material is selected from perylenes, quinacridones, phthalocyanines, isoindolines, dioxazines (that is, triphenedioxazines), 1,4-diketopyrrolopyrroles, anthrapyrimidines, anthanthrones, flavanthrones, indanthrones, perinones, pyranthrones, thioindigos, 4,4′-diamino-1,1′-dianthraquinonyl, as well as substituted derivatives thereof, and mixtures thereof.

Mixtures of any of the previously described inorganic particles and/or organic particles can also be used.

If desired, the particles described above can be formed into nanoparticles. In certain embodiments, the nanoparticles are formed in situ during formation of an aqueous dispersion of polymer-enclosed particles, as described in more detail below. In other embodiments, however, the nanoparticles are formed prior to their incorporation into such an aqueous dispersion. In these embodiments, the nanoparticles can be formed by any of a number of various methods known in the art. For example, the nanoparticles can be prepared by pulverizing and classifying the dry particulate material. For example, bulk pigments such as any of the inorganic or organic pigments discussed above, can be milled with milling media having a particle size of less than 0.5 millimeters (mm), or less than 0.3 mm, or less than 0.1 mm. The pigment particles typically are milled to nanoparticle sizes in a high energy mill in one or more solvents (either water, organic solvent, or a mixture of the two), optionally in the presence of a polymeric grind vehicle. If necessary, a dispersant can be included, for example, (if in organic solvent) SOLSPERSE® 32000 or 32500 available from Lubrizol Corporation, or (if in water) SOLSPERSE® 27000, also available from Lubrizol Corporation. Other suitable methods for producing the nanoparticles include crystallization, precipitation, gas phase condensation, and chemical attrition (i.e., partial dissolution).

As indicated, in certain embodiments, the colorant present in the compositions of the present invention comprises a dispersion, such as an aqueous dispersion or an organic dispersion, of polymer-enclosed particles, such as nanoparticles. As used herein, a “dispersion of polymer-enclosed particles” refers to a continuous phase in which is dispersed discreet “composite microparticles” that comprise a particle, such as a nanoparticle, and a resin enclosing the nanoparticle.

Example aqueous dispersions of polymer-enclosed particles, which are suitable for use in the present invention, and methods for making them, are identified in United States Patent Application Publication 2005-0287348 A1 at [0036] to [0050] and U.S. patent application Ser. No. 10/876,031 at [0054] to [0090], the cited portion of which being incorporated herein by reference. The methods described therein comprise (A) providing a mixture, in an aqueous medium, of (i) particles, (ii) one or more polymerizable, ethylenically unsaturated monomers; and/or (iii) a mixture of one or more polymerizable unsaturated monomers with one or more polymers; and/or (iv) one or more polymers, and then (B) subjecting the mixture to high stress shear conditions in the presence of an aqueous medium. If present, the ethylenically unsaturated monomers then can be polymerized under free radical conditions as described below.

The aqueous medium used in the foregoing method generally is exclusively water. However, for some monomer and/or polymer systems, it can be desirable to also include a minor amount of inert organic solvent for example to assist in lowering the viscosity of the polymer to be dispersed. Typically, the amount of organic solvent present is less than 20 weight percent, based on total weight of the dispersion. Examples of suitable organic solvents which can be incorporated for this purpose include, but are not limited to propylene glycol monobutyl ether, ethylene glycol monohexyl ether, ethylene glycol monobutyl ether, n-butanol, benzyl alcohol, and mineral spirits.

When included, the polymerizable, ethylenically unsaturated monomers can include any of the polymerizable ethylenically, unsaturated monomers, including vinyl monomers known in the art. Non-limiting examples of such monomers include unsaturated carboxylic acid functional group-containing monomers, ethylenically unsaturated monomers free of carboxylic acid functional groups, and ethylenically unsaturated, beta-hydroxy ester functional monomers, such as those materials specifically listed in paragraphs [0056]-[0058] of U.S. patent application Ser. No. 10/876,031.

As previously mentioned, in the foregoing methods, the particles can be admixed with one or more polymers, such as one or more polymers selected from acrylic polymers, polyurethane polymers, polyester polymers, polyether polymers, silicon-based polymers, co-polymers thereof, and mixtures thereof. Such polymers may also include crosslinking agents. Specific examples of suitable polymers include those identified in paragraphs [0061]-[0076] of U.S. patent application Ser. No. 10/876,031.

As indicated, in these methods, the particles can also be admixed, optionally in the presence of an aqueous medium, with a mixture of one or more of the polymerizable, ethylenically unsaturated monomers described above, and one or more of the polymers described above. Likewise, if desired, mixtures of crosslinking agents can be used, as well as mixtures of crosslinking agents with the one or more polymers and/or the one or more ethylenically unsaturated monomers described above.

In certain embodiments, the one or more monomers and/or one or more polymers are present in the dispersion in an amount of 10 to 80 weight percent, such as 20 to 70 weight percent, or, in some cases, 30 to 60 weight percent based on total weight of solids present in the dispersion.

In the foregoing methods, after the particles are admixed with the one or more polymerizable monomers and/or the one or more polymers as discussed above, the admixture is subjected to high stress shear conditions to particularize the admixture into microparticles. The high stress shear can be accomplished by any of the high stress shear techniques well known in the art.

As used herein, the term “high stress shear conditions” is meant to include not only high stress techniques, such as by the liquid-liquid impingement techniques discussed in detail below, but also high speed shearing by mechanical means. It should be understood that, if desired, any mode of applying stress to the admixture can be utilized so long as sufficient stress is applied to achieve particularization of the admixture into microparticles and the requisite particle size distribution.

The admixture can be subjected to the appropriate stress by use of a MICROFLUIDIZER® emulsifier which is available from Microfluidics Corporation in Newton, Mass. The MICROFLUIDIZER® high-pressure impingement emulsifier is described in detail in U.S. Pat. No. 4,533,254, which is hereby incorporated by reference. The device consists of a high-pressure (up to about 1.4×105 kPa (20,000 psi)) pump and an interaction chamber in which emulsification takes place. The pump forces the admixture, typically in aqueous medium, into the chamber where it is split into at least two streams which pass at very high velocity through at least two slits and collide, resulting in the formation of small particles, i.e., the admixture is “particularized”. Generally, the pre-emulsion admixture is passed through the emulsifier at a pressure of between about 3.5×104 and about 1×105 kPa (5,000 and 15,000 psi). Multiple passes can result in smaller average particle size and a narrower range for the particle size distribution. When using the aforesaid MICROFLUIDIZER® emulsifier, stress is applied by liquid-liquid impingement as has been described. As mentioned above, other modes of applying stress to the pre-emulsification admixture can be utilized so long as sufficient stress is applied to achieve the requisite particle size distribution. For example, one alternative manner of applying stress would be the use of ultrasonic energy.

As discussed above, in the foregoing methods, the particles can be admixed either with a mixture of one or more polymerizable, ethylenically unsaturated monomers, or with one or more polymerizable, ethylenically unsaturated monomers and one or more polymers. If either case, the polymerizable ethylenically unsaturated monomers (and polymers if used) are blended with the particles, optionally in the presence of an aqueous medium, to form a pre-emulsion admixture. The pre-emulsion admixture is then subjected to high stress conditions in the presence of an aqueous medium as described above to particularize the admixture thereby forming microparticles dispersed in the aqueous medium. The polymerizable species within each particle, if present, typically are subsequently polymerized (i.e. the polymer is formed in situ, typically under suitable free-radical polymerization conditions as described below) under conditions sufficient to produce composite microparticles (each having a first organic or polymeric phase, and a second nanoparticulate phase) which are stably dispersed in the aqueous medium.

In some cases, a surfactant or dispersant can be present to stabilize the dispersion. The surfactant usually is present when the organic component referred to above is mixed into the aqueous medium prior to particularization into microparticles. Alternatively, the surfactant can be introduced into the medium at a point just after the microparticles have been formed.

In order to conduct the polymerization of the ethylenically unsaturated monomers in the presence of the particles (and the polymer when used), a free radical initiator typically is present. Both water-soluble and oil soluble initiators can be used. Examples of water-soluble initiators include ammonium peroxydisulfate, potassium peroxydisulfate and hydrogen peroxide. Examples of oil soluble initiators include t-butyl hydroperoxide, dilauryl peroxide and 2,2′-azobis(isobutyronitrile). Generally, the reaction is carried out at a temperature ranging from 20° to 80° C. The polymerization can be carried out in either a batch or a continuous process. The length of time necessary to carry out the polymerization can range from 10 minutes to 6 hours, provided that the time is sufficient to form a polymer in situ from the one or more ethylenically unsaturated monomers.

Once the microparticles have been formed and the polymerization process, if any, is complete, the resultant product is a stable dispersion of microparticles in an aqueous medium which can contain some organic solvent. Some or all of the organic solvent can be removed via reduced pressure distillation at a temperature, for example, of less than 40° C. As used herein, by “stable dispersion” or “stably dispersed” is meant that the microparticles neither settle nor coagulate nor flocculate from the aqueous medium upon standing.

In other embodiments, aqueous dispersions of polymer-enclosed particles, which are suitable for use in the present invention, are made by a method comprising (1) providing a mixture, in an aqueous medium, of (i) particles, (ii) a polymerizable ethylenically unsaturated monomer, and (iii) a water-dispersible polymerizable dispersant, and (2) polymerizing the ethylenically unsaturated monomer and polymerizable dispersant to form polymer-enclosed color-imparting particles comprising a water-dispersible polymer. Such methods are described in detail in U.S. patent application Ser. No. 11/337,062, filed Jan. 20, 2006, which is incorporated herein by reference.

In these methods, the polymerizable dispersant may comprise any polymerizable material that is water-dispersible and which, upon polymerization with the ethylenically unsaturated monomer, produces polymer-enclosed particles comprising a water-dispersible polymer. As used herein, the term “water-dispersible” means that a material may be dispersed in water without the aid or use of a surfactant.

In certain embodiments, the polymer-enclosed color-imparting particles used in the present invention comprise, for example, a polymer selected from acrylic polymers, polyurethane polymers, polyester polymers, polyether polymers, silicon-based polymers, co-polymers thereof, and mixtures thereof, so long as the polymer or mixture of polymers is water-dispersible. Such polymers can be produced by any suitable method known to those skilled in the art to which the present invention pertains and includes those polymers disclosed in U.S. patent application Ser. No. 10/876,031 at [0061] to [0076].

In certain embodiments of these methods, the polymerizable dispersant is a friable polymer. As used herein, the term “friable polymer” refers to a polymer that is easily pulverized at ambient conditions. In certain embodiments, the polymerizable dispersant comprises a water-dispersible, polymerizable polyester polyurethane having terminal ethylenic unsaturation, as described in paragraphs [0056]-[0079] of U.S. patent application Ser. No. 11/337,062.

In these methods, the water-dispersible polymerizable dispersant is capable is dispersing itself and other materials, including the ethylenically unsaturated monomers, in the aqueous medium without the need for surfactants and/or high shear conditions. As a result, the foregoing method for making an aqueous dispersion of polymer-enclosed particles is particularly suitable in situations where use of the high stress shear conditions described U.S. patent application Ser. No. 10/876,031 at [0081] to [0084] is not desired or feasible. Therefore, in certain embodiments, the foregoing methods do not include the step of subjecting the mixture of particles, polymerizable ethylenically unsaturated monomer, and water-dispersible polymerizable dispersant to high stress shear conditions.

In addition, the foregoing method enables the formation of nanoparticles in situ, rather than requiring the formation of nanoparticles prior to preparation of the aqueous dispersion. In these methods, particles having an average particle size of 1 micron or more, after being mixed with the ethylenically unsaturated monomer and the water-dispersible polymerizable dispersant in the aqueous medium, may be formed into nanoparticles (i.e., the nanoparticles are formed in situ). In certain embodiments, the nanoparticles are formed by subjecting the aqueous medium to pulverizing conditions. For example, the particles can be milled with milling media having a particle size of less than 0.5 millimeters, or less than 0.3 millimeters, or, in some cases, less than 0.1 millimeters. In these embodiments, the particles can be milled to nanoparticle size in a high energy mill in the presence of the aqueous medium, the polymerizable ethylenically unsaturated monomer, and the water-dispersible polymerizable dispersant. If desired, another dispersant can be used, such as SOLSPERSE 27000, available from Avecia, Inc.

As indicated, the foregoing methods include the step of free-radically polymerizing the ethylenically unsaturated monomer and polymerizable dispersant to form polymer-enclosed particles comprising a water-dispersible polymer. In certain embodiments, at least a portion of the polymerization occurs during formation of nanoparticles, if applicable. Also, a free radical initiator may be used. Both water and oil soluble initiators can be used, including those described earlier. In many cases, the reaction is carried out at a temperature ranging from 20° to 80° C. The polymerization can be carried out in either a batch or a continuous process. The length of time necessary to carry out the polymerization can range from, for example, 10 minutes to 6 hours, provided that the time is sufficient to form a polymer in situ from the one or more ethylenically unsaturated monomers.

Once the polymerization process is complete, the resultant product is a stable dispersion of polymer-enclosed particles in an aqueous medium which can contain some organic solvent.

In certain embodiments, the polymer-enclosed particles are present in the aqueous dispersions of the present invention in an amount of at least 10 weight percent, or in an amount of 10 to 80 weight percent, or in an amount of 25 to 50 weight percent, or in an amount of 25 to 40 weight percent, with weight percents being based on weight of total solids present in the dispersion.

In certain embodiments, the dispersed polymer-enclosed particles have a maximum haze of 10%, or, in some cases, a maximum haze of 5%, or, in yet other cases, a maximum haze of 1%, or, in other embodiments, a maximum haze of 0.5%. As used herein, “haze” is determined by ASTM D1003.

The haze values for the polymer-enclosed particles described herein are determined by first having the particles, such as nanoparticles, dispersed in a liquid (such as water, organic solvent, and/or a dispersant, as described herein) and then measuring these dispersions diluted in a solvent, for example, butyl acetate, using a Byk-Gardner TCS (The Color Sphere) instrument having a 500 micron cell path length. Because the % haze of a liquid sample is concentration dependent, the % haze as used herein is reported at a transmittance of about 15% to about 20% at the wavelength of maximum absorbance. An acceptable haze may be achieved for relatively large particles when the difference in refractive index between the particles and the surrounding medium is low. Conversely, for smaller particles, greater refractive index differences between the particle and the surrounding medium may provide an acceptable haze.

In the foregoing methods, upon reaction of the ethylenically unsaturated monomer with the polymerizable dispersant, polymer-enclosed particles are formed, which, as previously indicated, the inventors believe results in a phase barrier that physically prevents the particles, particularly nanoparticles, from re-agglomerating within the aqueous dispersion. As a result, the foregoing methods of the present invention result in an aqueous dispersion of particles, such as nanoparticles, wherein reagglomeration of the nanoparticles is minimized or avoided altogether. In certain embodiments, the compositions of the present invention comprise an organic dispersion of polymer-enclosed color imparting particles. Such an organic dispersion can be formed from the conversion of an aqueous dispersion of polymer-enclosed color-imparting particles.

In certain embodiments of the present invention, therefore, an aqueous dispersion of polymer-enclosed color-imparting particles is converted into an organic dispersion of polymer-enclosed color-imparting particles prior and such an organic dispersion is included in the compositions of the present invention. This conversion can be accomplished by, for example, diluting an aqueous dispersion of polymer-enclosed color-imparting particles of the type previously described with an organic solvent, particularly a water-miscible organic solvent, such as a polar protic organic solvent, wherein the solvent is added in an amount sufficient to produce a dispersion wherein the continuous phase comprises predominantly organic solvent, i.e., the amount of water present in the aqueous dispersions is less than 20 weight percent, such as less than 10 weight percent, or, in some cases, less than 5 weight percent, or, in yet other cases, less than 2 weight percent, with the weight percents being based on the total weight of the dispersion. If desired, the amount of water present in the dispersion can be further reduced via a distillation process.

As used herein, the term “water-miscible organic solvent” refers to organic solvents that, at the conditions of use, are miscible with water in a reasonably wide concentration range. Examples include, without limitation, N-methylpyrrolidone and tetrahydrofuran.

In certain embodiments, the water miscible organic solvent comprises a polar protic solvent, which are those solvents wherein a hydrogen atom is attached to an electronegative atom, such as oxygen. In other words, polar protic organic solvents are compounds that can be represented by the general formula ROH, wherein R is an organic radical. The polarity of the polar protic solvents stems from the bond dipole of the O—H bond. Examples of polar protic organic solvents, which are suitable for use in the present invention, are methanol, ethanol, isopropanol, butanol, and acetic acid, as well as propylene glycol and ethylene glycol.

In certain embodiments of the compositions of the present invention, the colorant comprises a radiation diffraction material comprising an ordered periodic array of particles held in a matrix. Such colorants are described in, for example, U.S. Pat. No. 6,894,086 at col. 3, line 52 to col. 11, line 53, the cited portion of which being incorporated herein by reference.

In certain embodiments, a photosensitive composition and/or photochromic composition, which reversibly alters its color when exposed to one or more light sources, can be used in the compositions of the present invention. Photochromic and/or photosensitive compositions can be activated by exposure to radiation of a specified wavelength. When the composition becomes excited, the molecular structure is changed and the altered structure exhibits a new color that is different from the original color of the composition. When the exposure to radiation is removed, the photochromic and/or photosensitive composition can return to a state of rest, in which the original color of the composition returns. In certain embodiments, the photochromic and/or photosensitive composition can be colorless in a non-excited state and exhibit a color in an excited state. Full color-change can appear within milliseconds to several minutes, such as from 20 seconds to 60 seconds. Example photochromic and/or photosensitive compositions include photochromic dyes.

In certain embodiments of the present invention, a photosensitive composition and/or photochromic composition is associated with and/or at least partially bound to, such as by covalent bonding, a polymer and/or polymeric materials of a polymerizable component. Example photosensitive compositions and/or photochromic compositions and methods for making them are identified in United States Published Patent Application No. 2006-0014099 A1, which is incorporated herein by reference.

In addition to the previously described colorants, the personal care compositions of the present invention comprise other materials, one or more of which may be included depending upon the desired end use.

For example, in certain embodiments, the personal care compositions of the present invention comprise a protein. Proteins are often used to provide durability in personal care compositions. In addition, proteins have been used to impart beneficial coating effects, such as manageability and strength to hair, to moisturize skin and hair, and to provide film formation to improve the appearance of skin and hair. Suitable proteins include silk proteins, such as the acrylated silk proteins described in U.S. Pat. No. 5,747,105, the relevant portion of which being incorporated herein by reference, chemically modified proteins, such as those described in U.S. Pat. No. 6,296,860, the relevant portion of which being incorporated herein by reference, enzymatically digested silk protein and water-soluble silk protein (useful in the area of wound care), as described in U.S. Pat. Nos. 5,382,431 and 6,175,053, the relevant portions of both of which being incorporated herein by reference, as well as protein polymers, such as is described in United States Published Patent Application No. 2005/0142094 A1, incorporated herein by reference.

In certain embodiments, the personal care compositions of the present invention comprise a liposome. As used herein, the term “liposome” refers to an artificial microscopic vesicle having an aqueous core enclosed in one or more phospholipid layers, used to convey vaccines, drugs, enzymes, or other substances to target cells or organs. In certain embodiments, such liposomes comprise components such as water and one or more ingredients capable of forming lipid bilayer vesicles that can hold one or more functional or active ingredient(s). Non-limiting examples of ingredients capable of forming lipid bilayer vesicles include: phospholipids, hydrogenated phosphatidylcholine, lecithin, cholesterol and sphingolipids. Non-limiting examples of functional or active ingredients that can be delivered from liposomes include: vitamins and their derivatives, antioxidants, proteins and peptides, keratolytic agents, bioflavinoids, terpenoids, phytochemicals, and extracts of plant, marine or fermented origin. In certain embodiments, liposomes include, for example: a) lipoid liposome 0003 (composed of water and lecithin and glycerin); b) lipoid liposome 0300 (composed of water and phosphatidylcholine), c) lipoid liposome 0111 (composed of water, ginkgo balboa leaf extract, denatured alcohol, hydrogenated lecithin and cholesterol) d) anti-irritant liposomes (composed of water, cola acuminata seed extract, bisabolol and phospholipids), e) vitamin C and E liposomes (composed of water, phospholipids, tocopheryl acetate and ascorbyl palmitate), f) firming liposomes (composed of water, butylene glycol, pyrus malus (apple) fruit extract, phospholipids, tocopheryl acetate and carbomer) and g) moisturizing liposomes (composed of water, sodium PCA, tocopheryl acetate, xanthan gum, arginine, lysine, glycine and proline).

In certain embodiments, the personal care compositions of the present invention comprise a personal care active ingredient. As used herein, the term “personal care active ingredient” refers to a compound that has a cosmetic effect and/or a medical effect on the body, for example, the skin, hair, or nails. In certain embodiments, the personal care active ingredient is present in the personal care compositions of the present invention in an amount of from 0.001% to 20% by weight of the composition, such as 0.01% to 10%, or, in some cases, 0.1% to 5% by weight, based on the total weight of the composition.

For example, suitable personal care active ingredients for skin care compositions include, but are not limited to, antioxidants (e.g., tocopheryl and ascorbyl derivatives); bioflavinoids, terpenoids, synthetics of bioflavinoids and terpenoids and the like; vitamins and vitamin derivatives; hydroxyl- and polyhydroxy acids and their derivatives (e.g., AHAs and BHAs and their reaction products); peptides and polypeptides and their derivatives (e.g., glycopeptides and lipophilized peptides, heat shock proteins and cytokines); enzymes and enzymes inhibitors and their derivatives (e.g., proteases, MMP inhibitors, catalases, CoEnzyme Q10, glucose oxidase and superoxide dismutase (SOD)); amino acids and their derivatives; bacterial, fungal and yeast fermentation products and their derivatives (e.g., mushrooms, algae and seaweed and their derivatives); phytosterols and plant and plant part extracts; phospholipids and their derivatives; anti-dandruff agents (e.g., zinc pyrithione); and sunscreen agents (e.g., ethylhexyl methoxycinnamate, avobenzone, and phenyl benzimidazole sulfonic acid).

In certain embodiments of the skin care compositions of the present invention, the personal care active ingredient comprises a Vitamin B3 component, panthenol, Vitamin E, Vitamin E acetate, retinoid, retinol, retinyl, propionate, retinyl palmitate, retinoic acid, Vitamin C, theobromine, α-hydroxyacid, farnesol, phytrantriol, salicylic acid, palmityl peptapeptide-3 or a mixture thereof. In certain embodiments, the Vitamin B3 compound is niacinamide. In yet other embodiments, the vitamin B3 compound is tocopherol nicotinate. Exemplary derivatives of the foregoing vitamin B3 compounds include nicotinic acid esters (e.g., non-vasodilating esters of nicotinic acid, nicotinyl amino acids, nicotinyl alcohol esters of carboxylic acids, and nicotinic acid N-oxide and niacinamide N-oxide). Suitable esters of nicotinic acid include nicotinic acid esters of C₁-C₂₂, such as C₁-C₁₆, or, in some cases, C₁-C₆ alcohols. The alcohols may be straight-chain or branched chain, cyclic or acyclic, saturated or unsaturated (including aromatic), and substituted or unsubstituted. The esters are specifically non-vasodilating. Non-vasodilating esters of nicotinic acid include tocopherol nicotinate and inositol hexanicotinate. Although these compounds are well known to those in the art, a more complete description of vitamin B3 compounds is provided by WO 98/22085.

In some embodiments, the retinoid skin care active ingredient comprises retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid). These compounds are well known in the art and are commercially available from a number of sources (e.g., Sigma-Aldrich Chemical Company, and Boehringer Mannheim). Exemplary retinoids include retinol, retinyl palmitate, retinyl acetate, retinyl propionate, retinal, retinoic propionate, retinoic acid and combinations thereof. In certain embodiments, the retinoid is included as a substantially pure material, while in other embodiments, it is an extract obtained by suitable physical and/or chemical isolation from natural (e.g., plant) sources. In certain embodiments, the retinoid comprises from 0.005% to 2%, such as 0.01% to 12%, by weight of the personal care composition. In other embodiments, the personal care composition comprises retinol. In certain embodiments, the retinol comprises from 0.01% to 0.15% by weight of the personal care composition. In yet other embodiments, the personal care composition comprise retinol esters. In certain embodiments, the retinol esters comprise from 0.01% to 2% by weight of the personal care composition.

In addition to the personal care active ingredients noted above, in certain embodiments, the personal care composition of the present invention comprise a physiologically acceptable carrier and/or excipient. In certain embodiments, such personal care compositions comprise a dermatologically acceptable carrier suitable for topical application to the skin, nails, mucous membranes, and/or hair within which the other materials are incorporated to enable the materials to be delivered to the site of application (i.e., the target site) at an appropriate concentration. Thus, the carrier acts as a diluent, dispersant, solvent or the like for the personal care compositions and the colorant which ensures that they can be applied to and distributed evenly over the selected target site at an appropriate concentration.

In certain embodiments, an effective amount of one or more compounds described herein is included in personal care compositions suitable for application to keratinous materials such as nails and hair, including but not limited to, those useful as hair spray compositions, hair styling compositions, hair shampooing and/or conditioning compositions, compositions applied for the purpose of hair growth regulation and compositions applied to the hair and scalp for the purpose of treating seborrhoea, dermatitis and/or dandruff.

In certain embodiments, an effective amount of one or more compounds described herein is included in personal care compositions suitable for topical application to the skin, teeth, nails or hair. In certain embodiments, these compositions are in the form of creams, lotions, gels, suspensions dispersions, microemulsions, nanodispersions, microspheres, hydrogels, emulsions (e.g., oil-in-water and water-in-oil, as well as multiple emulsions) and multilaminar gels and the like (See e.g., Schlossman et al., The Chemistry and Manufacture of Cosmetics, [1998]). In certain embodiments, these compositions are formulated as aqueous or silicone compositions, while in other embodiments they are formulated as emulsions of one or more oil phases in an aqueous continuous phase, and in still further embodiments, are an aqueous phase in an oil phase.

The type of carrier utilized in the personal care compositions of the present invention depends on the type of product form desired. In certain embodiments, the carrier is a solid, while in other embodiments, it is semi-solid or liquid. Suitable carriers include liquids, as well as semi-solids (e.g., creams, lotions, gels, sticks, suppositories, ointments, pastes, sprays and mousses). In certain embodiments, the carrier itself is inert, while in other embodiments, it possesses benefits of its own. In some embodiments, the carrier is applied directly to the teeth, skin, nails and/or hair, while in other embodiments, it is applied via a woven or non-woven wipe or cloth. In certain embodiments, it is provided as a patch, mask, wrap, or another inert substrate. In yet further embodiments, the carrier is aerosolized or otherwise sprayed or pumped onto the skin and/or hair.

In certain embodiments of the personal care compositions of the present invention, the carrier comprises water, propylene glycol, ethanol, propanol, glycerol, butylene glycol, or polyethylene glycol, as well as any suitable combination thereof. In certain embodiments, the carrier also contains at least one dermatologically acceptable, hydrophilic diluent. Suitable hydrophilic diluents include water, organic hydrophilic diluents (e.g., C₂-C₁₀, such as C₂-C₆, or, in some cases, C₃-C₆ monohydric alcohols) and low molecular weight glycols and polyols (e.g., propylene glycol, polyethylene glycol polypropylene glycol, glycerol, butylene glycol, 1,2,4-butanetriol, sorbitol, 1,2,6-hexametriol, pentylene glycol, hexylene glycol, sorbitol esters, ethoxylated ethers, propoxylated ethers) and combinations thereof. In certain embodiments, the diluent is a liquid, such as water. In certain embodiments, the personal care composition comprises at least 20% by weight of the hydrophilic diluent.

In certain embodiments, the carrier that is present in the personal care compositions of the present invention comprises an emulsion comprising a hydrophilic phase, such as an aqueous phase, and a hydrophobic phase (e.g., a lipid, oil or oily material). As will be appreciated, the hydrophilic phase is dispersed in the hydrophobic phase, or vice versa, to form respectively hydrophilic or hydrophobic dispersed and continuous phases, depending on the composition of ingredients. In certain embodiments, the emulsion comprises an oil-in-water emulsion or a water-in-oil emulsion, such as a water-in-silicone emulsion (e.g., in a triple or other multi-phase emulsion). In certain embodiments, oil-in-water emulsions comprise from 1% to 60%, such as from 1% to 30% of the dispersed hydrophobic phase, and from 1% to 99% or, in some cases, from 10% to 90% of the continuous hydrophilic phase, while in some alternative embodiments, water-in-oil emulsions comprise from 1% to 98%, or from 40% to 90% of the dispersed hydrophilic phase and from 1% to 50%, such as from 1% to 30% of the continuous hydrophobic phase.

In certain embodiments of the personal care compositions of the present invention, the carrier also includes one or more components that facilitate penetration through the upper stratum corneum barrier to the deeper skin layers, such as may be the case when employing the compositions of the present invention as a tattoo composition, wherein the composition is injected into the skin of the person receiving the tattoo. Examples of penetration enhancers include, but are not limited to, propylene glycol, azone, ethoxydiglycol, dimethyl isosorbide, urea, ethanol, dimethyl sulfoxide, microemulsions, liposomes and nanoemulsions.

In certain embodiments, the personal care compositions of the present invention comprise a humectant. In certain embodiments, the humectant is present in such personal care composition in an amount ranging from 0.01 to 20 percent by weight, such as 0.1 to 15 percent by weight, or, in some cases, 0.5 to 10 percent by weight, with the weight percents being based on the total weight of the composition. Suitable humectants include, but are not limited to, polyhydric alcohols, sorbitol, glycerol, urea, betaine, D or DL panthenol, calcium pantothenate, royal jelly, panthetine, pantotheine, panthenyl ethyl ether, pangamic acid, pyridoxin, pantoyl lactose Vitamin B complex, sodium pyrrolidone carboxylic acid, hexane-1,2,6,-triol, guanidine or its derivatives, and mixtures thereof.

Examples of suitable polyhydric alcohol humectants include, for example, polyalkylene glycols, such as alkylene polyols and their derivatives, including propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, pentylene glycol, hexylene glycol, butylene glycol (e.g., 1,3-butylene glycol), hexane triol (e.g., 1,2,6-hexanetriol), trimethylol propane, neopentyl glycol, glycerine, ethoxylated glycerine, propane-1,3 diol, propoxylated glycerine and mixtures thereof. The alkoxylated derivatives of any of the above polyhydric alcohols are also suitable. In some embodiments, the polyhydric alcohol is selected from glycerine, butylene glycol, propylene glycol, pentylene glycol, hexylene glycol, dipropylene glycol, polyethylene glycol, hexane triol, ethoxylated glycerine and propoxylated glycerine and mixtures thereof.

Additional suitable humectants include sodium 2-pyrrolidone-5-carboxylate (NaPCA), guanidine, glycolic acid, glycolate salts (e.g., ammonium and quaternary alkyl ammonium), lactic acid, lactate salts (e.g., ammonium and quaternary alkyl ammonium), aloe vera in any of its variety of forms (e.g., aloe vera gel), hyaluronic acid and derivatives thereof (e.g., salt derivatives such as sodium hyaluronate), lactamide monoethanolamine, acetamide monoethanolamine, urea, betaine, panthenol and derivatives thereof, and mixtures thereof.

In some embodiments, at least part of a humectant is incorporated in the form of an admixture with a particulate cross-linked hydrophobic acrylate or methacrylate copolymer, which is present in an amount of, for example, from 0.1 to 10 percent, which can be added either to the aqueous or disperse phase. As known in the art (see e.g., WO96/03964), this copolymer is particularly valuable for reducing shine and controlling oil, while helping to provide effective moisturization benefits.

In certain embodiments, the personal care compositions embodied as an oil-in-water or water-in-oil emulsion comprise from 0.05 to 20 percent by weight, such as from 1 to 15 percent by weight, or, in some cases, from 2 to 10 percent by weight, or, in yet other embodiments from 2 to 5 percent by weight of a dermatologically acceptable emollient. Emollients tend to lubricate the skin, increase the smoothness and suppleness of the skin, prevent or relieve dryness of the skin and/or protect the skin. Emollients are typically water-immiscible, oily or waxy materials and emollients with high molecular weights can confer aesthetic properties to a topical composition. A wide variety of suitable emollients are known and find use herein. Specific examples of suitable emollients include, but are not limited to, (i) straight and branched chain hydrocarbons having from about 7 to about 40 carbon atoms, such as mineral oils, dodecane, squalane, cholesterol, hydrogenated polyisobutylene, isohexadecane, isoeicosane, isooctahexacontane, isohexapentacontahectane, and the C₇-C₄₀ isoparaffins, which are C₇-C₄₀ branched hydrocarbons, such as isopentacontaoctactane, petrolatum and mixtures thereof; (ii) C₁-C₃₀ fatty acid esters of C₁-C₃₀ carboxylic acids, C₁₂₋₁₅ alkyl benzoates and of C₂-C₃₀ dicarboxylic acids, such as isononyl isononanoate, isostearyl neopentanoate, isodecyl octanoate, isodecyl isononanoate, tridecyl isononanoate, myristyl octanoate, octyl pelargonate, octyl isononanoate, myristyl myristate, myristyl neopentanoate, myristyl octanoate, isopropyl myristate, myristyl propionate, isopropyl stearate, isopropyl isostearate, methyl isostearate, behenyl behenate, dioctyl maleate, diisopropyl adipate, and diisopropyl dilinoleate and mixtures thereof; (iii) C₁-C₃₀ mono- and poly-esters of sugars and related materials derived from a sugar or polyol moiety and one or more carboxylic acid moieties, such as glucose tetraoleate, the galactose tetraesters of oleic acid, the sorbitol tetraoleate, sucrose tetraoleate, sucrose pentaoleate, sucrose hexaoleate, sucrose heptaoleate, sucrose octaoleate, sorbitol hexaester, cottonseed oil, and/or soybean oil fatty acid esters of sucrose; (iv) vegetable oils and hydrogenated vegetable oils, such as safflower oil, grapeseed oil, coconut oil, cottonseed oil, menhaden oil, palm kernel oil, palm oil, peanut oil, soybean oil, rapeseed oil, linseed oil, rice bran oil, pine oil, nut oil, sesame oil, sunflower seed oil, partially and fully hydrogenated oils from the foregoing sources and mixtures thereof; and (v) soluble or colloidally-soluble moisturizing agents, such as hyaluaronic acid and, chondroitin sulfate, heparan sulfate, and starch-grafted sodium polyacrylates.

In certain embodiments, the personal care compositions of the present invention also comprise an emulsifier and/or surfactant, generally to, for example, help disperse and suspend the disperse phase within the continuous phase. Surfactants find particular use in, for example, products intended for skin and/or hair cleansing. Known and/or conventionally used surfactants (See e.g., WO 00/24372) find use in the personal care compositions of the present invention, provided that the selected agent is chemically and physically compatible with the other components of the composition and provides the desired characteristics. Suitable surfactants include non-silicone derived materials, silicone-derived materials, and mixtures thereof. In certain embodiments, the personal care compositions of the present invention comprise from 0.05 to 30 percent by weight, such as from 0.5 to 15 percent by weight, or, in some cases, from 1 to 10 percent by weight of surfactant, based on the total weight of the composition. As known to those skilled in the art, the exact surfactant or surfactant mixture chosen will depend upon the pH of the composition, the other components present and the desired final product aesthetics.

Among the nonionic surfactants that are useful herein are the condensation products of long chain alcohols (e.g., C₈₋₃₀ alcohols, with sugar or starch polymers, such as glycosides). Other useful nonionic surfactants include the condensation products of alkylene oxides with fatty acids (i.e., alkylene oxide esters of fatty acids). These materials have the general formula RCO(X)ₙOH wherein R is a C₁₀₋₃₀ alkyl group, X is —OCH₂CH₂— (i.e., derived from ethylene glycol or oxide) or —OCH₂CHCH₃— (i.e., derived from propylene glycol or oxide) and n is an integer from 6 to 200. Other nonionic surfactants are the condensation products of alkylene oxides with 2 moles of fatty acids (i.e., alkylene oxide diesters of fatty acids). These materials have the general formula RCO(X)ₙOOCR wherein R is a C₁₀₋₃₀ alkyl group, X is —OCH₂CH₂— (i.e., derived from ethylene glycol or oxide) or —OCH₂CHCH₃— (i.e., derived from propylene glycol or oxide) and n is an integer from 6 to 100. For example, in certain embodiments, the personal care compositions of the present invention comprise an emulsifier that is a fatty acid ester blend based on a mixture of sorbitan fatty acid ester and sucrose fatty acid ester, more specifically a blend of sorbitan stearate and sucrose cocoate. Even further suitable examples include a mixture of cetearyl alcohols and cetearyl glucosides.

Hydrophilic surfactants useful herein can alternatively or additionally include any of a wide variety of cationic, anionic, zwitterionic, and amphoteric surfactants such as those known in the art (See, e.g., McCutcheon's, Detergents and Emulsifiers and Detergents, North American [2003], and International Editions [1986], published by MC Publishing Co. and Allured Publishing Corporation; and U.S. Pat. Nos. 5,011,681, 4,421,769, and 3,755,560, herein incorporated by reference).

A variety of anionic surfactants are known in the art (See e.g., U.S. Pat. No. 3,929,678, herein incorporated by reference) and also find use in certain embodiments of the personal care compositions of the present invention. Examples of anionic surfactants include the alkoyl isethionates (e.g., C₁₂-C₃₀), alkyl and alkyl ether sulfates and salts thereof, alkyl and alkyl ether phosphates and salts thereof, alkyl methyl taurates (e.g., C₁₂-C₃₀), and soaps (e.g., substituted alkylamine and alkali metal salts, such as sodium or potassium salts) of fatty acids.

Amphoteric and zwitterionic surfactants are suitable for use in certain embodiments of the personal care compositions of the present invention, including those that are broadly described as derivatives of aliphatic secondary and tertiary amines in which the aliphatic radical can be straight or branched chain and wherein one of the aliphatic substituents contains from 8 to 22 carbon atoms, such as C₈-C₁₈) and one contains an anionic water solubilizing group (e.g., carboxy, sulfonate, sulfate, phosphate, or phosphonate). Examples include, but are not limited to, alkyl imino acetates and iminodialkanoates and aminoalkanoates, imidazolinium, as well as those selected from the group consisting of betaines, sultaines, hydroxysultaines, and branched and unbranched alkanoyl sarcosinates, and mixtures thereof.

In certain embodiments, the personal care compositions of the present invention that are embodied as an emulsion include a silicone containing emulsifier or surfactant. These silicone emulsifiers are typically organically modified organopolysiloxanes, also known to those skilled in the art as silicone surfactants. Useful silicone emulsifiers include dimethicone copolyols, which are polydimethyl siloxanes which have been modified to include polyether side chains, such as polyethylene oxide chains, polypropylene oxide chains, mixtures of these chains and polyether chains containing moieties derived from both ethylene oxide and propylene oxide. Other examples include alkyl-modified dimethicone copolyols (i.e., compounds which contain C₂-C₃₀ pendant side chains). Still other useful dimethicone copolyols include materials having various cationic, anionic, amphoteric, and zwitterionic pendant moieties.

In certain embodiments, the personal care compositions of the present invention comprise a polymeric thickening agent, such as those having a number average molecular weight of greater than 20,000, such as greater than 50,000, or, in some cases, greater than 100,000. In certain embodiments, the personal care compositions of the present invention comprise from 0.01 to 10 percent by weight, such as from 0.1 to 8 percent by weight, or, in some cases, from 0.25 to 5 percent by weight of a polymeric thickening agent, or mixtures thereof.

Examples of polymer thickening agents suitable for use in certain embodiments of the personal care compositions of the present invention include non-ionic thickening agents and anionic thickening agents or mixtures thereof. Suitable non-ionic thickening agents include polyacrylamide polymers, crosslinked poly(N-vinylpyrrolidones), polysaccharides, natural or synthetic gums, polyvinylpyrrolidone and polyvinylalcohol. Suitable anionic thickening agents include acrylic acid/ethyl acrylate copolymers, carboxyvinyl polymers and crosslinked copolymers of alkyl vinyl ethers and maleic anhydride. As an example, Noveon sells a thickener under the trademark of CARBOPOL™ resins or mixtures thereof. In some embodiments, suitable CARBOPOL™ resins are hydrophobically modified, while in other embodiments, suitable resins include those described in WO98/22085, or mixtures thereof.

In certain embodiments, the personal care compositions of the present invention comprise at least one silicone oil phase. In certain embodiments, such silicone oil phase(s) comprise from 0.1 to 20 percent by weight, such as from 0.5 to 10 percent by weight, or, in some cases, from 0.5 to 5 percent by weight of the composition. In some embodiments, the silicone oil phase comprises one silicone component, while in alternative embodiments, the silicone oil phase comprises more than one silicone component.

In certain embodiments, silicone components are fluids, including straight chain, branched and cyclic silicones. Suitable silicone fluids useful herein include silicones inclusive of polyalkyl siloxane fluids, polyaryl siloxane fluids, cyclic and linear polyalkylsiloxanes, polyalkoxylated silicones, amino and quaternary ammonium modified silicones, polyalkylaryl siloxanes or a polyether siloxane copolymer and mixtures thereof. In some embodiments, the silicone fluids are volatile, while in other embodiments, the silicone fluids are non-volatile. In certain embodiments, the silicone fluid used in the present invention is selected from silicone fluids having a weight average molecular weight in the range from 100 to 50,000, such as from 200 to about 40,000.

The silicone fluids used in certain embodiments of the personal care compositions of the present invention have a viscosity ranging from about 0.65 to about 600,000 mm² s⁻¹, such as from 0.65 to 10,000 mm² s⁻¹ at 25° C. The viscosity can be measured by means of a glass capillary viscometer as set forth in Dow Corning Corporate Test Method CTM0004, Jul. 29, 1970. Suitable polydimethyl siloxanes that find use in the present invention include commercially available compounds (e.g., from General Electric Company and Dow Corning). In additional embodiments, essentially non-volatile polyalkylarylsiloxanes (e.g., polymethylphenylsiloxanes, having viscosities of about 0.65 to 30,000 mm² s⁻¹ at 25° C.; available from General Electric Company and Dow Corning) find use in the present invention. Cyclic polydimethylsiloxanes suitable for use herein often have a ring structure incorporating from 3 to 7 (CH₃)₂SiO moieties, such as 5 or more.

In certain embodiments, the personal care compositions of the present invention comprise a silicon gum or a mixture of silicones including a silicone gum. Typically, silicone gums have a viscosity at 25° C. in excess of 1,000,000 mm² s⁻¹. Silicone gums that find use in the present invention include dimethicones (e.g., those described in U.S. Pat. No. 4,152,416, herein incorporated by reference). Specific examples of silicone gums include, but are not limited to, polydimethylsiloxane, (polydimethylsiloxane)(methylvinylsiloxane) copolymer, poly(dimethylsiloxane)-(diphenyl)(methylvinylsiloxane) copolymer and mixtures thereof.

In certain embodiments, a silicone gum is incorporated into the composition as part of a silicone gum-fluid blend. In these embodiments, the silicone gum often constitutes from 5 to 40 percent by weight, such as from 10 to 20 percent by weight of the silicone gum-fluid blend. Suitable silicone gum-fluid blends for use herein include, but are not limited to, mixtures comprising: (i) a silicone having a molecular weight of from 200,000 to 4,000,000 and selected from dimethiconol, fluorosilicone and dimethicone and mixtures thereof; and (ii) a carrier which is a silicone fluid, the carrier having a viscosity from 0.65 mm² s⁻¹ to 100 mm² s⁻¹ at 25° C. mm, wherein the ratio of (i) to (ii) is from 10:90 to 20:80, and wherein said silicone gum-based component has a final viscosity of from 100 mm² s⁻¹ to 100,000 mm² s⁻¹ at 25° C., such as from 500 mm² s⁻¹ to 10,000 mm² s⁻¹ at 25° C.

Additional silicone components suitable for use in the silicone oil phase of some embodiments of the present invention include crosslinked polyorganosiloxane polymers, including those that are dispersed in a fluid carrier. In certain embodiments, the crosslinked polyorganosiloxane polymers, together with their carrier (if present) comprise from 0.1 to 20 percent by weight, such as from 0.5 to 10 percent by weight, or, in some cases, from 0.5 to 5 percent by weight of the personal care composition. Suitable polymers include, for example, those comprising polyorganosiloxane polymers, such as methyl vinyl dimethicone, methyl vinyl diphenyl dimethicone and methyl vinyl phenyl methyl diphenyl dimethicone, crosslinked by a crosslinking agent, as described in WO98/22085.

Another class of silicone components suitable for use in certain embodiments of the personal care compositions of the present invention are polydiorganosiloxane-polyoxyalkylene copolymers containing at least one polydiorganosiloxane segment and at least one polyoxyalkylene segment, as described in WO98/22085. Suitable polydiorganosiloxane-polyalkylene copolymers are available commercially under the tradename BELSIL™ from Wacker-Chemie GmbH. An example of a copolymer fluid blend for use herein includes Dow Corning DC3225C which has the CTFA designation Dimethicone/Dimethicone copolyol.

In certain embodiments, the personal care compositions of the present invention comprise an organic sunscreen. In certain embodiments, suitable radioprotectives (e.g., sunscreens) exhibit UVA-absorbing properties and/or UVB-absorbing properties. The exact amount of the sunscreen active will vary, depending upon the desired Sun Protection Factor (“SPF”) of the composition, as well as the desired level of UV protection. SPF is a commonly used indicator of photoprotection of a sunscreen against erythema. The SPF is defined as a ratio of the ultraviolet energy required to produce minimal erythema on protected skin to that required to produce the same minimal erythema on unprotected skin in the same individual. In certain embodiments, the sunscreen is present in the personal care composition in an amounts of 2 to 20 percent by weight, such as from 4 to 14 percent by weight of the personal care composition. Suitable sunscreens include, but are not limited to, those approved for use in the United States, Japan, Europe and Australia. In certain embodiments, the personal care compositions of the present invention comprise an SPF of 2 to 30, such as 4 to 30, and, in some cases, 4 to 15.

In certain embodiments, the personal care compositions of the present invention include a UVA absorbing sunscreen that absorbs ultraviolet radiation having a wavelength of from 320 nanometers to 400 nanometers. Suitable UVA absorbing sunscreen actives include those selected from dibenzoylmethane derivatives (See e.g., Lowe and Shaath (eds.), Sunscreens: Development, Evaluation, and Regulatory Aspects, Marcel Dekker, Inc [1990]), anthranilate derivatives (e.g., methylanthranilate and homomethyl, 1-N-acetylanthranilate), and mixtures thereof. In certain embodiments, the UVA absorbing sunscreen active is present in an amount suitable to provide broad spectrum UVA protection either independently, or in combination with, any other UV protective actives present in the composition.

Suitable UVA sunscreen actives include dibenzoylmethane sunscreen actives and their derivatives. They include, but are not limited to, those selected from 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropylbenzoylmethane, 4-(1,1-dimethylethyl)-4′-methoxydibenzoyl-methane, 2-methyl-5-isopropyl-4′-methoxydibenzoylmethane, 2-methyl-5-tert-butyl-4′-methoxy-dibenzoylmethane, 2,4-dimethyl-4′-methoxydibenzoylmethane, 2,6-dimethyl-4′-tert-butyl-4′methoxydibenzoylmethane, and mixtures thereof. In certain embodiments, the dibenzoyl sunscreen actives include those selected from 4-(1,1-dimethylethyl)-4′-methoxydibenzoylmethane, 4-isopropyldibenzoylmethane, and mixtures thereof.

The sunscreen active 4-(1,1-dimethylethyl)-4′-methoxydibenzoyl-methane, which is also known as butyl methoxydibenzoylmethane or avobenzone, is commercially available under the names of PARSOL® 1789 (Givaudan Roure (International) S. A.), and EUSOLEX® 9020 (Merck & Co., Inc). The sunscreen 4-isoproplydibenzoylmethane, which is also known as isopropyldibenzoylmethane, is commercially available from Merck under the name of EUSOLEX® 8020.

In certain embodiments, the personal care compositions of the present invention include one or more UVB sunscreen actives that absorb UV radiation having a wavelength of from 290 nanometers to 320 nanometers. The compositions comprise an amount of the UVB sunscreen active that is safe and effective in providing UVB protection either independently, or in combination with, any other UV protective actives present in the compositions. In some embodiments, the compositions comprise from 0.1 to 20 percent by weight, such as from 0.1 to 12 percent by weight, or, in some cases, from 0.5 to 8 percent by weight of each UVB absorbing organic sunscreen, or as mandated by any relevant regulatory authority.

A variety of UVB sunscreen actives find use in the present invention, including, but not limited to organic sunscreen actives described in U.S. Pat. Nos. 5,087,372, 5,073,371, and 5,073,372, each of which being incorporated herein by reference. Additional sunscreens that find use in the present invention include those described in U.S. Pat. Nos. 4,937,370 and 4,999,186, both of which being incorporated herein by reference. In certain embodiments, the UVB sunscreen active is selected from 2-ethylhexyl-2-cyano-3,2-ethylhexyl N,N-dimethyl-p-aminobenzoate, p-amino-benzoic acid, oxybenzone, homomethyl salicylate, octyl salicylate, 4,4′-methoxy-t-butyldibenzoylmethane, 4-isopropyl dibenzoylmethane, 3-benzylidene camphor, 3-(4-methylbenzylidene)camphor, 3-diphenylacrylate, 2-phenyl-benzimidazole-5-sulphonic acid (PBSA), cinnamate esters and their derivatives such as 2-ethylhexyl-p-methoxycinnamate and octyl-p-methoxycinnamate, salicylate esters and their derivatives such as TEA triethanolamine salicylate, ethylhexyl saliycyilate, octyldimethyl para-aminobenzoic acid PABA, camphor derivatives and their derivatives, and mixtures thereof. Examples of organic sunscreen actives include 2-ethylhexyl-2-cyano-3,3-diphenylacrylate, 2-phenyl-benzimidazole-5-sulphonic acid (PBSA), octyl-p-methoxycinnamate, and mixtures thereof. Salt and acid neutralized forms of the acidic sunscreens are also find use herein.

In certain embodiments of the personal care compositions of the present invention, an agent is added to stabilize the UVA sunscreen(s) to prevent photo-degradation upon exposure to UV radiation, thereby maintaining UVA protection efficacy. A wide range of compounds can be used to provide these stabilizing properties and should be chosen to complement both the UVA sunscreen and the composition as a whole. Suitable stabilizing agents include, but are not limited to, those described in WO 00/06110, and U.S. Pat. Nos. 5,972,316, 5,968,485, 5,935,556, 5,827,508, each of which being incorporated herein by reference. Specific, but non-limiting, examples of stabilizing agents for use in the present invention include 2-ethylhexyl-2-cyano-3,3-diphenylacrylate, ethyl-2-cyano-3,3-diphenylacrylate, 2-ethylhexyl-3,3-diphenylacrylate, ethyl-3,3-bis(4-methoxyphenyl)acrylate, diethylhexyl 2,6 napthalate and mixtures thereof (Symrise Chemical Company).

In certain embodiments of the personal care compositions of the present invention, an agent is added to enhance the resistance of the composition to being washed off by water or rubbed off. Examples of such materials include, but are not limited to, acrylates/C₁₂₋₂₂ alkylinethacrylate copolymer, acrylate/acrylate copolymer, dimethicone, dimethiconol, graft-copoly (dimethylsiloxane/iI-butyl methacrylate), lauryl dimethicone, PVP/Hexadecane copolymer, PVP/Eicosene copolymer, tricontanyl PVP and trimethoxysiloxysiliacate.

In addition to organic sunscreens, some embodiments of personal care compositions of the present invention additionally comprise inorganic physical sunblocks, such as any of those described in CTFA International Cosmetic Ingredient Dictionary, 6^{th} Edition, [1995], pp. 1026-28 and 1103; Sayre et al., J. Soc. Cosmet. Chem., 41:103-109 [1990]. In certain embodiments, inorganic physical sunblocks, such as zinc oxide and/or titanium dioxide, and mixtures thereof are used.

In certain embodiments of the personal care products of the present invention, physical sunblocks are present in an amount from 0.5 to 20 percent by weight, such as from 0.51 to 10 percent by weight, and, in some cases, from 0.5 to 5 percent by weight of the composition. Anatase, rutile, and/or amorphous titanium dioxide find use in certain embodiments of the present invention. In certain embodiments, physical sunblock particles (e.g., titanium dioxide and zinc oxide), are uncoated, while in alternative embodiments, the particles are coated with a variety of materials including but not limited to amino acids, aluminium compounds, such as alumina, aluminium stearate, aluminium laurate, and the like; carboxylic acids and their salts (e.g., stearic acid and its salts); phospholipids (e.g., lecithin); organic silicon compounds; inorganic silicon compounds (e.g., silica and silicates), and mixtures thereof.

In certain embodiments, the personal care compositions of the present invention also comprise a preservative, such as, but are not limited to, pentylene glycol, ethylene diamine tetra acetate (“EDTA”) and its salts, chlorhexidine (and its diacetate, dihydrochloride, digluconate derivatives), 1,1,1-trichloro-2-methyl-2-propanol, parachloro metaxylenol, polyhexamethylenebiguanide hydrochloride, dehydroacetic acid, diazolidinyl urea, 2,4-dichlorobenzyl alcohol, 4,4-dimethyl-1,3-oxazolidine, formaldehyde, glutaraldehyde, dimethylidantoin, imidazolidinyl urea, 5-chloro-2-methyl-4-isothiazolin-3-one, ortho-phenylphenol, 4-hydroxybenzoic acid and its (methyl-, ethyl-, propyl-, isopropyl-, butyl-, isobutyl-) esters (also known as parabens), salts, trichlosan, 2-phenoxyethanol, phenyl mercuric acetate, borate, nitrate, quaternium-15, salicilate, salicylic acid and its salts, calcium, sorbic acid and its salts, iodopropanyl butylcarbamate, calcium sorbate, zinc pyrithione, benzyl alcohol, 5-bromo-Snitro-1,3-dioxane, 2-bromo-2-nitropropane-1,3-diol, benzoic acid and its salts, sulfites, bisulfites, and benzalkonium chloride, phenoxyethanol and chloroxylenol, and diazolidinyl urea.

In certain embodiments, a variety of optional ingredients such as neutralizing agents, perfumes and perfume solubilizing agents are included in the personal care compositions of the present invention. Neutralizing agents suitable for use in neutralizing acidic group containing hydrophilic gelling agents herein include sodium hydroxide, potassium hydroxide, ammonium hydroxide, monoethanolamine, diethanolamine, amino methyl propanol, tris-buffer and triethanolamine.

In certain embodiments, other optional materials are included in the present invention, including any of the various functional and/or active ingredients known to those skilled in the art, such as keratolytic agents; water-soluble or solubilizable preservatives at a level of, for example, from 0.1 to 5 percent, such as methyl, ethyl, propyl and butyl esters of hydroxybenzoic acid, benzyl alcohol, DMDM hydantoin iodopropanyl butylcarbanate available under the trade name GLYDANT PLUS™ (Lonza), EDTA, EUXYL® K400, BROMOPOL™ (2-bromo-2-nitropropane-1,3-diol) and phenoxypropanol; anti-bacterials such as IRGASAN® and phenoxyethanol (specifically at levels of from about 0.1 to about 5 percent); soluble or colloidally-soluble moisturizing agents such as hyaluaronic acid and chondroitin sulfatestarch-grafted sodium polyacrylates, such as SANWET® IM-1000, IM-1500 and IM-2500 available from Celanese Superabsorbent Materials, Portsmith, Va., USA and described in U.S. Pat. No. 4,076,663; vitamins such as vitamin A, vitamin C, vitamin E, vitamin K and derivatives thereof and building blocks thereof; such as phytantriol; and vitamin K and components thereof such as the fatty alcohols such as dodecatrienol; alpha and beta hydroxyacids; aloe vera; sphingosines and phytosphingosines, cholesterol; skin whitening agents; N-acetyl cysteine; colouring agents; antibacterial agents such as TCC/TCS, also known as triclosan and trichlorocarbon; perfumes and perfume solubilizers. Examples of alpha hydroxy acids include glycolic acid, lactic acid, malic acid, and citric acid (whether derived synthetically or from natural sources and whether used alone or in combination), and their esters or relevant buffered combinations, such as glycolic acid in conjunction with ammonium glycolate. Other examples of alpha-hydroxy acids include: alpha-hydroxy ethanoic acid, alpha-hydroxyoctanoic acid, alpha-hydroxycaprylic acid, and hydroxycaprylic acid, mixed fruit acid, tri-alpha hydroxy fruit acids, triple fruit acid, sugar cane extract, alpha hydroxy and botanical comprise, 1-alpha hydroxy acid and glycomer in crosslinked fatty acids alpha nutrium. Specific examples of alpha hydroxy acids include glycolic acid and lactic acid, used at, for example, levels of up to about 10 percent.

In certain embodiments, the pH of the personal care compositions herein is in the range from about 3.5 to about 10, such as from 4 to 8, or, in some cases, from 5 to 7, wherein the pH of the final composition is adjusted by addition of acidic, basic or buffer salts as necessary, depending upon the composition of the forms and the pH requirements of the compounds.

Those skilled in the art will appreciate the various techniques for preparing the personal care compositions of the present invention, any of which may be employed herein. In some cases, an aqueous phase and/or the oil phase are prepared separately, with materials of similar phase partitioning being added in any suitable order. For emulsion final products, the two phases are combined with vigorous stirring and/or homogenization as necessary to reduce the size of the internal phase droplets. Any ingredients in the formulation with high volatility, or which are susceptible to hydrolysis or decomposition at high temperatures, are preferably added with gentle stirring towards the end of the process, and/or at the post emulsification stage, if applicable. As known to those skilled in the art, dosage frequency and amount depends upon the desired performance criteria.

As previously indicated, in certain embodiments the personal care compositions of the present invention comprise an oral hygiene composition. Such compositions have traditionally been in the form of clear solutions. Increasingly, however, there has been consumer demand for new and interesting oral hygiene compositions, such as those having a pleasing appearance.

In certain embodiments, the oral hygiene compositions of the present invention comprise a water phase and an oil phase. In addition to water, the water phase may comprise, for example, a humectant, a sweetener, and/or a thickening agent. Suitable humectants include any of those described earlier.

Any food grade and/or pharmaceutically acceptable sweetener maybe used in the water phase, including saccharin, fructose, xylitol, saccharin salts, thaumatin, aspartame, D-tryptophan, dihydrochalcones, acesulfame and cyclamate salts, especially sodium cyclamate and sodium saccharin, and combinations thereof.

Any food grade or pharmaceutically acceptable thickening agent may be used, such as those comprising a hydrophilic colloid which forms a gel when added to the water phase and the thickening agent is preferably dispersed in a carrier. One example is xanthan gum dispersed in glycerin. Other acceptable thickening agents are polymeric polyester compounds, natural gums (e.g. gum karaya, gum arabic, gum tragacanth), carrageenan, hydroxymethyl cellulose, methyl cellulose, carboxymethylcellulose, arrowroot powder, starches, particularly corn starch and potato starch and the like, either alone or with a carrier such as glycerin, polyethylene glycol or combinations thereof, and the like. The thickening agent may comprise combinations of these hydrophilic colloids. Generally, the thickening agent will comprise up to 5 percent hydrophilic colloid and 95.0 percent to 99.9 percent carrier, such as from 0.1 percent to 1.0 percent hydrophilic colloid and 99.0 percent to 99.9 percent carrier, based on the total weight of the thickening agent/carrier dispersion.

Calcium lactate and calcium lactate salts are white crystalline powders, and any calcium lactate or salt that is acceptable for food or pharmaceutical applications may be used. Calcium lactate and calcium lactate salt are effective against tartar buildup and, thus, can serve to provide a personal care active ingredient in embodiments where the personal care composition is in the form of oral hygiene compositions, and also provides a source of calcium for the personal care composition. Calcium lactate is available from Purac—North America, Lincolnshire, Ill., U.S.A. under the trade name PURACAL®.

In certain embodiments of the oral hygiene compositions of the present invention, the oil phase comprises a surfactant, such as any of the surfactants described earlier.

Flavoring agents useful in the oral hygiene compositions of the present invention include any food grade or pharmaceutically acceptable flavoring agent. Preferably, the flavoring agent comprises natural flavoring oils, including those selected from the group consisting of oil of peppermint, oil of wintergreen, oil of spearmint, clove bud oil, parsley oil, eucalyptus oil and the like. Combinations of oils can also be used. The flavoring agents may comprise compounds selected from the group consisting of menthol, menthane, anethole, methyl salicylate, eucalyptol, cassia, 1-methyl acetate, sage, eugenol, oxanone, α-irisone, marjoram, lemon, orange, propenyl guaethol acetyl, cinnamon, vanilla, thymol, linalool, cinnamaldehyde glycerol acetal and the like, and combinations thereof. The flavoring agent may comprise combinations of natural flavoring oils and other flavoring agents such as the compounds identified above. The flavoring agents are, in certain embodiments, added to the oil phase.

Examples of cooling agents useful for the invention include those comprising menthol, N-substituted p-menthane-3-carboxamides (such as N-ethyl p-methane-3-carboxamide), 3,1-methoxy propane 1,2-diol and the like. Warming agents include capsicum and nicotinate esters, such as benzyl nicotinate. Numbing agents include benzocaine, lidocaine, clove bud oil, and ethanol. Any food grade and/or pharmaceutically acceptable flavoring agent may be included in the oral hygiene compositions of the present invention. In certain embodiments, the flavoring agent comprises natural flavoring oils, including those selected from the group consisting of oil of peppermint, oil of wintergreen, oil of spearmint, clove bud oil, parsley oil, eucalyptus oil and the like, including combinations thereof. The flavoring agents may comprise compounds selected from the group consisting of menthol, menthane, anethole, methyl salicylate, eucalyptol, cassia, 1-methyl acetate, sage, eugenol, oxanone, α-irisone, marjoram, lemon, orange, propenyl guaethol acetyl, cinnamon, vanilla, thymol, linalool, cinnamaldehyde glycerol acetal and the like, and combinations thereof. The flavoring agent may comprise combinations of natural flavoring oils and other flavoring agents, such as the compounds identified above. The flavoring agents are, in certain embodiments, added to the oil phase, however, flavoring agents can be added with the additives, fillers and other ingredients whether or not flavoring agents are incorporated into the humectant phase.

As will be appreciated from the foregoing description, therefore, the present invention is also directed to methods for treating a keratinous substrate, comprising applying to at least a portion of the substrate a treatment composition comprising a colorant, wherein the colorant comprises (i) a radiation diffraction material comprising an ordered periodic array of particles held in a matrix, (ii) a dispersion of polymer-enclosed nanoparticles, or (iii) a mixture thereof. As used herein, the term “effective amount” means an amount sufficient to achieve the sought after cosmetic effect and/or medical effect.

In still other respects, the present invention is directed to methods for protecting a keratinous substrate from environmental damage, comprising applying to at least a portion of the substrate a composition comprising a colorant, wherein the colorant comprises (i) a radiation diffraction material comprising an ordered periodic array of particles held in a matrix, (ii) a dispersion of polymer-enclosed nanoparticles, or (iii) a mixture thereof.

Other embodiments of the present invention are directed to compositions that are applied to plants, trees, seeds, agricultural lands, such as grazing lands, crop lands and the like; turf-covered land areas, e.g., lawns, golf courses, athletic fields, etc., and other land areas such as forests and the like. As a result, in certain embodiments, the present invention is directed to agrochemical compositions comprising a colorant, wherein the colorant comprises: (a) a radiation diffraction material comprising an ordered periodic array of particles held in a matrix, (b) a dispersion of polymer-enclosed nanoparticles, or (c) a mixture thereof. As used herein, the term “agrochemical composition” refers to synthetic chemical compositions that are suitable for use in agriculture and, for purposes of the present invention include, an agricultural active ingredient. As used herein, the term “agricultural active ingredient” refers to a compound that serves to aid or contribute to the germination of a seed and/or growth of a seedling or plant. For purposes of the present invention, the term “agricultural active ingredient” includes, for example, fertilizers, pesticides, fungicides, nematocides, rodenticides, bird repellants, herbicides, miticides, insecticides, growth regulators, plant nutrients, and the like.

In certain embodiments, therefore, the present invention is directed to agrochemical compositions comprising a colorant as aforementioned and at least one active ingredient selected from a fertilizer, a herbicide, an insecticide, a fungicide, and/or a bactericide.

Fertilizers suitable for use in the agrochemical compositions of the present invention include, for example, macronutrients and micronutrients. Suitable macronutrients include, but are not limited to, nitrogen containing substances, such as ammonium nitrate, monoammonium phosphate, ammonium phosphate sulfate, ammonium sulfate, ammonium phosphatenitrate, diammonium phosphate, ammoniated single superphosphate, ammoniated triple superphosphate, nitric phosphates, ammonium chloride, aqua ammonia, ammonia-ammonium nitrate solutions, calcium ammonium nitrate, calcium nitrate, calcium cyanamide, sodium nitrate, urea, urea-formaldehyde, urea-ammonium nitrate solution, nitrate of soda potash, potassium nitrate, amino acids, proteins, nucleic acids; phosphorous containing substances, such as superphosphate (single, double and/or triple), phosphoric acid, ammonium phosphate, ammonium phosphate sulfate, ammonium phosphate nitrate, diammonium phosphate, ammoniated single superphosphate, ammoniated single superphosphate, ammoniated triple superphosphate, nitric phosphates, potassium pyrophosphates, sodium pyrophosphate, nucleic acid phosphates; X-potassium chloride, potassium sulfate, potassium gluconate, sulfate of potash magnesia, potassium carbonate, potassium acetate, potassium citrate, potassium hydroxide, potassium manganate, potassium phosphate, potassium molybdate, potassium thiosulfate, potassium zinc sulfate; calcium containing substances, such as calcium ammonium nitrate, calcium nitrate, calcium cyanamide, calcium acetate, calcium acetylsalicylate, calcium borate, calcium borogluconate, calcium carbonate, calcium chloride, calcium citrate, calcium ferrous citrate, calcium glycerophosphate, calcium lactate, calcium oxide, calcium pantothenate, calcium proprionate, calcium saccharate, calcium sulfate, calcium tartrate; magnesium containing substances, such as magnesium oxide, dolomite, magnesium acetate, magnesium bensoate, magnesium bisulfate, magnesium borate, magnesium chloride, magnesium citrate, magnesium nitrate, magnesium phosphate, magnesium salicylate, magnesium sulfate; sulfur containing substances, such as ammonium sulfate, ammonium phosphate sulfate, calcium sulfate, potassium sulfate, magnesium sulfate, sulfuric acid, cobalt sulfate, copper sulfate, ferric sulfate, ferrous sulfate, sulfur, cysteine, methionine.

Suitable micronutrients include, for example, zinc containing substances, such as zinc oxide, zinc acetate, zinc bensoate, zinc chloride, zinc citrate, zinc nitrate, zinc salicylate, ziram; iron containing substances, such as ferric chloride, ferric citrate, ferric fructose, ferric glycerophosphate, ferric nitrate, ferric oxide (saccharated), ferrous chloride, ferrous citrate ferrous fumarate, ferrous gluconate, ferrous succinate; manganese containing substances, such as manganese acetate, manganese chloride, manganese nitrate, manganese phosphate; copper containing substances, such as cupric acetate, cupric butyrate, cupric chlorate, cupric chloride, cupric citrate, cupric gluconate, cupric glycinate, cupric nitrate, cupric salicylate, cuprous acetate, cuprous chloride; boron containing substances, such as calcium borate, potassium borohydride, borax, boron trioxide, potassium borotartrate, potassium tetraborate, sodium borate, sodium borohydride, sodium tetraborate; molybdenum containing substances, such as molybdic acid, calcium molybdate, potassium molybdate, sodium molybdate; cobalt containing substances, such as cobaltic acetate, cobaltous acetate, cobaltous chloride, cobaltous oxalate, cobaltous potassium sulfate, cobaltous sulfate.

Herbicides suitable for use in the agrochemical compositions of the present invention include, for example: isourea plant growth regulators, such as N-methoxycarbonyl-N′-4-methylphenylcarbamoylethylisourea and 1-(4-chlorophenylcarbamoyl)-3-ethoxycarbonyl-2-methylisourea; other types of plant growth regulators, such as sodium naphthaleneacetate, 1,2-dihydropyridazine-3,6-dione and gibberellins; triazine herbicides, such as 2-methylthio-4,6-bisethylamino-1,3,5-triazine, 2-chloro-4,6-bisethylamino-1,3,5-triazine, 2-methoxy-4-ethylamino-6-isopropylamino-1,3,5-triazine, 2-chloro-4-ethylamino-6-isopropylamino-s-triazine, 2-methylthio-4,6-bis(isopropylamino)-s-triazine and 2-methylthio-4-ethylamino-6-isopropylamino-s-triazine; phenoxy herbicides, such as 2,4-dichlorophenoxyacetic acid and methyl, ethyl and butyl esters thereof, 2-chloro-4-methylphenoxyacetic acid, 4-chloro-2-methylphenoxyacetic acid and ethyl 2-methyl-4-chlorophenoxybutylate; diphenylether herbicides, such as 2,4,6-trichlorophenyl-4′-nitrophenylether, 2,4-dichlorophenyl-4′-nitrophenylether and 3,5-dimethylphenyl-4′-nitrophenylether; urea herbicides such as 3-(3,4-dichlorophenyl)-1-methoxy-1-methyl urea, 3-(3,4-dichlorophenyl)-1,1-dimethylurea and 3-(4-chlorophenyl)-1,1-dimethyl urea; carbamate herbicides, such as 3-methoxycarbonylaminophenyl-N-(3-methylphenyl)carbamate, isopropyl-N-(3-chlorophenyl)carbamate and methyl-N-(3,4′-dichlorophenyl)carbamate; uracil herbicides such as 5-bromo-3-sec-butyl-6-methyluracil and 1-cyclohexyl-3,5-propyleneuracil; thiolcarbamate herbicides, such as S-(4-chlorobenzyl)-N,N-diethylthiolcarbamate, S-ethyl-N-cyclohexyl-N-ethylthiolcarbamate and S-ethyl-hexahydro-1H-azepine-1-carbothioate and S-ethyl-N,N-di-n-propylthiocarbamate; pyridinium herbicides, such as 1,1′-dimethyl-4,4′-bispyridinium dichloride; phosphoric herbicides, such as N-(phosphonomethyl)glycine; aniline herbicides such as α,α,α-trifluoro-2,6-dinitro-N,N-dipropyl-p-toluidine, 4-(methylsulfonyl)-2,6-dinitro-N,N-dipropylaniline and N³,N³-diethyl-2,4-dinitro-6-trifluoromethyl-1,3-phenylene diamine; acid anilide herbicides, such as 2-chloro-2′,6′-diethyl-N-(butoxymethyl)acetoanilide, 2-chloro-2′,6′-diethyl-N-(methoxymethyl)acetoanilide, and 3,4-dichloropropioneanilide; pyrazole herbicides, such as 1,3-dimethyl-4-(2,4-dichlorobenzoyl)-5-hydroxypyrazole and 1,3-dimethyl-4-(2,4-dichlorobenzoyl)-5-(p-toluenesulfonyloxy)pyrazole; 5-tert-butyl-3-(2,4-dichloro-5-isopropoxyphenyl)-1,3,4-oxadiazoline-2-one; 2-[N-isopropyl,N-(4-chlorophenyl)carbamoyl]-4-chloro-5-methyl-4-isooxazoline-3-one; 3-isopropylbenzo-2-thia-1,3-diazinone-(4)-2,4-dioxide and 3-(2-methylphenoxy)pyridazine.

Insecticides suitable for use in the agrochemical compositions of the present invention include, for example: phosphoric insecticides, such as O,O-diethyl O-(2-isopropyl-4-methyl-6-pyrimidinyl)phosphorothioate, O,O-diethyl S-2-[(ethylthio)ethyl]phosphorodithioate, O,O-dimethyl O-(3-methyl-4-nitrophenyl)thiophosphate, O,O-dimethyl S—(N-methylcarbamoylmethyl)phosphorodithioate, O,O-dimethyl S—(N-methyl-N-formylcarbamoylmethyl)phosphorodithioate, O,O-dimethyl S-2-[(ethylthio)ethyl]phosphorodithioate, O,O-diethyl S-2-[(ethylthio)ethyl]phosphorodithioate, O,O-dimethyl-1-hydroxy-2,2,2-trichloroethylphosphonate, O,O-diethyl-O-(5-phenyl-3-isooxazolyl)phosphorothioate, O,O-dimethyl O-(2,5-dichloro-4-bromophenyl)phosphorothioate, O,O-dimethyl O-(3-methyl-4-methylmercaptophenyl)thiophosphate, O-ethyl O-p-cyanophenyl phenylphosphorothioate, O,O-dimethyl-S-(1,2-dicarboethoxyethyl)phosphorodithioate, 2-chloro-(2,4,5-trichlorophenyl)vinyldimethyl phosphate, 2-chloro-1-(2,4-dichlorophenyl)vinyldimethyl phosphate, O,O-dimethyl O-p-cyanophenyl phosphorothioate, 2,2-dichlorovinyl dimethyl phosphate, O,O-diethyl O-2,4-dichlorophenyl phosphorothioate, ethyl mercaptophenylacetate O,O-dimethyl phosphorodithioate, S-[(6-chloro-2-oxo-3-benzooxazolinyl)methyl]O,O-diethyl phosphorodithioate, 2-chloro-1-(2,4-dichlorophenyl)vinyl diethylphosphate, O,O-diethyl 0-(3-oxo-2-phenyl-2H-pyridazine-6-yl)phosphorothioate, O,O-dimethyl S-(1-methyl-2-ethylsulfinyl)-ethyl phosphorothiolate, O,O-dimethyl S-phthalimidomethyl phosphorodithioate, O,O-diethyl S—(N-ethoxycarbonyl-N-methylcarbamoylmethyl)phosphorodithioate, O,O-dimethyl S-[2-methoxy-1,3,4-thiadiazol-5-(4H)-onyl-(4)-methyl]dithiophosphate, 2-methoxy-4H-1,3,2-benzooxaphosphorine 2-sulfide, O,O-diethyl O-(3,5,6-trichloro-2-pyridyl)phosphorothiate, O-ethyl O-2,4-dichlorophenyl thionobenzene phosphonate, S-[4,6-diamino-s-triazine-2-yl-methyl]O,O-dimethyl phosphorodithioate, O-ethyl O-p-nitrophenyl phenyl phosphorothioate, O,S-dimethyl N-acetyl phosphoroamidothioate, 2-diethylamino-6-methylpyrimidine-4-yl-diethylphosphorothionate, 2-diethylamino-6-methylpyrimidine-4-yl-dimethylphosphorothionate, O,O-diethyl O—N-(methylsulfinyl) phenyl phosphorothioate, O-ethyl S-propyl O-2,4-dichlorophenyl phosphorodithioate and cis-3-(dimethoxyphosphinoxy)N-methyl-cis-crotone amide; carbamate insecticides, such as 1-naphthyl N-methylcarbamate, S-methyl N-[methylcarbamoyloxy]thioacetoimidate, m-tolyl methylcarbamate, 3,4-xylyl methylcarbamate, 3,5-xylyl methylcarbamate, 2-sec-butylphenyl N-methylcarbamate, 2,3-dihydro-2,2-dimethyl-7-benzofuranylmethylcarbamate, 2-isopropoxyphenyl N-methylcarbamate, 1,3-bis(carbamoylthio)-2-(N,N-dimethylamino)propane hydrochloride and 2-diethylamino-6-methylpyrimidine-4-yl-dimethylcarbamate; and another insecticides, such as N,N-dimethyl N′-(2-methyl-4-chlorophenyl)formamidine hydrochloride, nicotine sulfate, milbemycin, 6-methyl-2,3-quinoxalinedithiocyclic S,S-dithiocarbonate, 2,4-dinitro-6-sec-butylphenyl dimethylacrylate, 1,1-bis(p-chlorophenyl) 2,2,2-trichloroethanol, 2-(p-tert-butylphenoxy)isopropyl-2′-chloroethylsulfite, azoxybenzene, di-(p-chlorophenyl)-cyclopropyl carbinol, di[tri(2,2-dimethyl-2-phenylethyl)tin]oxide, 1-(4-chlorophenyl)-3-(2,6-difluorobenzoyl)urea and S-tricyclohexyltin O,O-diisopropylphosphorodithioate. In addition, to the extent they are not identified above, suitable insecticides for use in the present invention are listed in U.S. Pat. No. 6,900,176 at col. 10, line 60 to col. 12, line 38, the cited portion of which being incorporated herein by reference.

Fungicides and bactericides suitable for use in the agrochemical compositions of the present invention include, for example: carbamate fungicides, such as 3,3′-ethylenebis(tetrahydro-4,6-dimethyl-2H-1,3,5-thiadiazine-2-thione), zinc or manganese ethylenebis(dithiocarbamate), bis(dimethyldithiocarbamoyl)disulfide, zinc propylenebis(dithiocarbamate), bis(dimethyldithiocarbamoyl)ethylenediamine; nickel dimethyldithiocarbamate, methyl 1-(butylcarbamoyl)-2-benzimidazolecarbamate, 1,2-bis(3-methoxycarbonyl-2-thioureido)benzene, 1-isopropylcarbamoyl-3-(3,5-dichlorophenyl)hydantoin, potassium N-hydroxymethyl-N-methyldithiocarbamate and 5-methyl-10-butoxycarbonylamino-10,11-dehydrodibenzo (b,f)azepine; pyridine fungicides, such as zinc bis(1-hydroxy-2(1H)pyridinethionate) and 2-pyridinethiol-1-oxide sodium salt; phosphorus fungicides, such as O,O-diisopropyl S-benzylphosphorothioate and O-ethyl S,S-diphenyldithiophosphate; phthalimide fungicides, such as N-(2,6p-diethylphenyl)phthalimide and N-(2,6-diethylphenyl)-4-methylphthalimide; dicarboxylmide fungicides, such as N-trichloromethylthio-4-cyclohexene-1,2-dicarboxylmide and N-tetrachloroethylthio-4-cyclohexene-1,2-dicarboxylmide; oxathine fungicides, such as 5,6-dihydro-2-methyl-1,4-oxathine-3-carboxanilido-4,4-dioxide and 5,6-dihydro-2-methyl-1,4-oxathine-3-carboxanilide; naphthoquinone fungicide, such as 2,3-dichloro-1,4-naphthoquinone, 2-oxy-3-chloro-1,4-naphthoquinone copper sulfate; pentachloronitrobenzene; 1,4-dichloro-2,5-dimethoxybenzene; 5-methyl-s-triazol(3,4-b)benzthiazole; 2-(thiocyanomethylthio)benzothiazole; 3-hydroxy-5-methylisooxazole; N-2,3-dichlorophenyltetrachlorophthalamic acid; 5-ethoxy-3-trichloromethyl-1,2,4-thiadiazole; 2,4-dichloro-6-(O-chloroanilino)-1,3,5-triazine; 2,3-dicyano-1,4-dithioanthraquinone; copper 8-quinolinate; polyoxine; validamycin; cycloheximide; iron methanearsonate; diisopropyl 1,3-dithiolane-2-iridene malonate; 3-allyloxy-1,2-benzoisothiazol-1,1-dioxide; kasugamycin; Blasticidin S; 4,5,6,7-tetrachlorophthalide; 3-(3,5-dichlorophenyl)-5-ethenyl-5-methyloxazolizine-2,4-dione; N-(3,5-dichlorophenyl)-1,2-dimethylcyclopropane-1,2-dicarboxylmide; S-n-butyl-5′-para-t-butylbenzyl-N-3-pyridyldithiocarbonylimidate; 4-chlorophenoxy-3,3-dimethyl-1-(1H,1,3,4-triazol-1-yl)-2-butanone; methyl-D,L-N-(2,6-dimethylphenyl)-N-(2′-methoxyacetyl)alaninate; N-propyl-N-[2-(2,4,6-trichlorophenoxy)ethyl]imidazol-1-carboxamide; N-(3,5-dichlorophenyl)succinimide; tetrachloroisophthalonitrile; 2-dimethylamino-4-methyl-5-n-butyl-6-hydroxypyrimidine; 2,6-dichloro-4-nitroaniline; 3-methyl-4-chlorobenzthiazol-2-one; 1,2,5,6-tetrahydro-4H-pyrrolo[3,2,1-i,j]quinoline-2-one; 3′-isopropoxy-2-methylbenzanilide; 1-[2-(2,4-dichlorophenyl)-4-ethyl-1,3-dioxorane-2-ylmethyl]-1H,1,2,4-triaz ol; 1,2-benzisothiazoline-3-one; basic copper chloride; basic copper sulfate; N′-dichlorofluoromethylthio-N,N-dimethyl-N-phenylsulfamide; ethyl-N-(3-dimethylaminopropyl)thiocarbamate hydrochloride; piomycin; S,S-6-methylquinoxaline-2,3-diyldithiocarbonate; complex of zinc and manneb; di-zinc bis(dimethyldithiocarbamate)ethylenebis (dithiocarbamate). In addition, to the extent they are not identified above, suitable fungicides and bactericides for use in the present invention are listed in U.S. Pat. No. 6,900,176 at col. 10, lines 1-59, the cited portion of which being incorporated herein by reference.

In addition, the agrochemical compositions of the present invention may include other components, including vitamins and cofactors, complexing agents, growth regulators, gum components, microbialstats, CSE components, and buffers, such as those identified in U.S. Pat. No. 5,797,976 at col. 7, line 44 to col. 9, line 17, the cited portion of which being incorporated herein by reference.

The agrochemical compositions of the present invention can be prepared in a manner known to those skilled in the art, for example by mixing the active ingredients and colorant with at least one solvent or diluent, emulsifier, dispersant and/or binder or fixative, water repellant, if appropriate desiccants and UV stabilizers. Suitable solvents and/or diluents, binders, plasticizers, fixatives for use in the agrochemical compositions of the present invention are identified in U.S. Pat. No. 6,900,176 at col. 15, line 6 to col. 16, lines 13, the cited portion of which being incorporated herein by reference.

The agrochemical compositions of the present invention may be used in, for example, the form of an aerosol, an unpressurized spray, for example pump and atomizer sprays, a nebulizer, a fogger, a foam, a gel, or a vaporizer product.

In certain embodiments of the present invention, the agrochemical composition is a coating composition, such as a seed coating composition, comprising a previously described agricultural active ingredient, a colorant as previously described, and a polymer. In the case of a seed coating composition, the polymer acts to hold the agricultural active ingredient on the seed. A variety of polymers can be used to prepare such coating compositions including, for example, proteins, polysaccarides, polyesters, polyethers, polyurethanes, polymers prepared from unsaturated monomers, and combinations thereof, so long as the polymer is capable of forming a film. Suitable polymers are identified in U.S. Pat. No. 6,329,319 at col. 3, line 16 to col. 5, line 47, the cited portion of which being incorporated herein by reference. The concentration of the polymer in the such coating composition ranges, in certain embodiments, from 0.01 to 10 weight percent, such as 0.1 to 5 weight percent, based on the total weight of the coating composition.

### EXAMPLE 1

### Polyurethane Dispersion

This example describes the preparation of a polyurethane dispersion that was subsequently used to the form the polyurethane/nanopigment dispersions of Examples 2 and 3. The polyurethane dispersion was prepared from the following mixture of ingredients in the amounts indicated:

| Ingredients | Weight (grams) |
|---|---|
| Charge I | |
| Poly (neopentylglycol adipate) ¹ | 780.0 |
| Dimethylolpropionic acid (DMPA) | 280.7 |
| Tri-ethylamine | 127.1 |
| Butylated hydroxytoluene | 2.5 |
| Triphenyl phosphate | 2.5 |
| Charge II | |
| Hydroxyethyl methacrylate (HEMA) | 116.7 |
| Butyl methacrylate | 791.2 |
| Charge III | |
| Methylene bis(4-cyclohexylisocyanate) | 1175.1 |
| Charge IV | |
| Butyl methacrylate | 57.5 |
| Charge V | |
| Deionized water | 4734.8 |
| Ethylenediamine | 49.2 |
| Dimethylethanolamine | 40.6 |
| Charge VI | |
| Butyl methacrylate | 50 |

¹ Poly (neopentylglycol adipate) having a number average molecular weight of 1000.

The polyurethane dispersion was prepared in a four neck round bottom flask equipped with an electronic temperature probe, mechanical stirrer, condenser, and a heating mantle. Charge I was stirred 5 minutes in the flask at a temperature of 90° C. Charge II was added and the mixture was cooled to 60° C. Charge III was added over a minute period. Charge IV was added and the resulting mixture was gradually heated to 90° C. over 45 minutes and then held at 90° C. for 3 hours. Charge V was stirred in a separate flask and heated to 80° C. 3000.0 g of the reaction product of Charges I, II, III, and IV was added to Charge V over 30 minutes. Charge VI was added and the resulting mixture was cooled to room temperature. The final product was a translucent emulsion with an acid value of 12.1, a Brookfield viscosity of 872 centipoise (spindle #3 at 30 rpm), a pH of 7.75, and a nonvolatile content of 29.4% as measured at 110° C. for one hour.

### EXAMPLE 2

### Polyurethane/Nanopigment Dispersion

This example describes the preparation of a nano-sized PB 15:3 phthalocyanine blue pigment dispersion. The dispersion was prepared from the following mixture of ingredients in the amounts indicated:

| Ingredients | Weight (grams) |
|---|---|
| Charge I | |
| Polyurethane dispersion of Example 1 | 4772.7 |
| Deionized water | 2304.5 |
| Hydroquinone methyl ether (MEHQ) | 1.36 |
| PB 15:3 pigment² | 700.0 |
| Shellsol OMS (Shell Chemical Co.) | 86.4 |
| Charge II | |
| Deionized water | 71.5 |
| t-Butyl hydroperoxide (70% aqueous solution) | 5.8 |
| Charge III | |
| Deionized water | 337.2 |
| Ferrous ammonium sulfate | 0.13 |
| Sodium metabisulfite | 8.18 |

²Commercially available from BASF Corp.

The ingredients of Charge I were mixed using a 4.5 inch Cowles blade attached to an air motor. The mixture was then pre-dispersed in a Premier Mill PSM-11 basket mill containing 353 mL of 1.2-1.7 mm Zirconox YTZ® milling media for 1.25 hours at 1000 fpm for the mix blades and 960 rpm pump speed and then recycled through an Advantis V15 Drais mill containing 500 mL of 0.3 mm Zirconox YTZ® grinding media in a one liter grinding chamber. The mixture was milled at 1400 rpm with a pump setting of 19 rpm for a total time of 15 hours. The progress of the milling was monitored by visually observing changes in the transparency of thin films of samples drawn down over black and white Leneta paper. Charge II was added and the resulting mixture was stirred 5 minutes. Charge III was added in two aliquots over 5 minutes. The final product was a cyan (Blue) liquid with a Brookfield viscosity of 356 centipoise (spindle #3 at 30 rpm), a pH of 7.29, and a nonvolatile content of 28.9% as measured at 110° C. for one hour.

### EXAMPLE 3

### Polyurethane/Nanopigment Dispersion

This example describes the preparation of a nano-sized PR 179 red pigment dispersion. The dispersion was prepared from the following mixture of ingredients in the amounts indicated:

| Ingredients | Weight (grams) |
|---|---|
| Charge I | |
| Polyurethane dispersion of Example 1 | 4772.7 |
| Deionized water | 2304.5 |
| Hydroquinone methyl ether (MEHQ) | 1.36 |
| PR 179 pigment³ | 700.0 |
| Shellsol OMS (Shell Chemical Co.) | 86.4 |
| Charge II | |
| Deionized water | 71.5 |
| t-Butyl hydroperoxide (70% aqueous solution) | 5.8 |
| Charge III | |
| Deionized water | 337.2 |
| Ferrous ammonium sulfate | 0.13 |
| Sodium metabisulfite | 8.18 |

³Commercially available from Ciba Pigments.

The ingredients of Charge I were mixed using a 4.5 inch Cowles blade attached to an air motor. The mixture was then pre-dispersed in a Premier Mill PSM-11 basket mill containing 353 mL of 1.2-1.7 mm Zirconox YTZ® milling media for 1.5 hours at 1000 fpm for the mix blades and 960 rpm pump speed and then recycled through an Advantis V15 Drais mill containing 500 mL of 0.3 mm Zirconox YTZ® grinding media in a one liter grinding chamber. The mixture was milled at 1260 fpm with a pump setting of 19 rpm for a total time of 15 hours. The progress of the milling was monitored by visually observing changes in the transparency of thin films of samples drawn down over black and white Leneta paper. Charge II was added and the resulting mixture was stirred 5 minutes. Charge III was added in two aliquots over 5 minutes. The final product was a red liquid with a Brookfield viscosity of 28.1 centipoise (spindle #3 at 30 rpm), a pH of 7.61, and a nonvolatile content of 28.2% as measured at 110° C. for one hour.

### EXAMPLE 4

### Concealing Powders

Concealing powders were produced from the materials in the following table using the process described below.

| Sample | Material | Grams |
|---|---|---|
| Cyan powder | Tint from example 2 | 0.5 |
| | Talc | 3 |
| | Water | 10 |
| | total | 13.5 |
| Red powder | Tint from example 3 | 0.5 |
| | talc | 3 |
| | water | 10 |
| | total | 13.5 |
| Pink Powder | Tint from example 3 | 0.1 |
| | talc | 3 |
| | water | 10 |
| | total | 13.1 |

The samples were prepared in scintillation vials and poured into aluminum trays that were placed in a 110° C. air circulating oven to dry for 1 hr. The dried samples were ground to powders with a mortar and pestle. The concealing powders were then rubbed onto chamois skin and found to hide fine wrinkles in a manner consistent with a commercially available control.

### EXAMPLE 5

### Polyurethane Dispersion

This example describes the preparation of a polyurethane dispersion that was subsequently used to form the respective polyurethane/nanopigment dispersions of Examples 6 to 8. The polyurethane dispersion was prepared from the following mixture of ingredients in the amounts indicated:

| Ingredients | Weight (grams) |
|---|---|
| Charge I | |
| Poly (butylene oxide)⁴ | 355.6 |
| Dimethylolpropionic acid (DMPA) | 119.2 |
| Tri-ethylamine | 54.0 |
| Butylated hydroxytoluene | 2.2 |
| Triphenyl phosphate | 1.1 |
| Charge II | |
| Hydroxyethyl methacrylate (HEMA) | 27.8 |
| Butyl methacrylate | 48.4 |
| Butyl acrylate | 319.2 |
| Charge III | |
| Methylene bis(4-cyclohexylisocyanate) | 558.9 |
| Charge IV | |
| Butyl methacrylate | 55.6 |
| Charge V | |
| Deionized water | 2086.3 |
| Diethanolamine | 20.2 |
| Ethylenediamine | 26.9 |
| Dimethylethanolamine | 19.7 |
| Charge VI | |
| Butyl methacrylate | 50.0 |

⁴Poly (butylene oxide) having a number average molecular weight of 1000.

The polyurethane dispersion was prepared in a four neck round bottom flask equipped with an electronic temperature probe, mechanical stirrer, condenser, and a heating mantle. Charge I was stirred 5 minutes in the flask at a temperature of 125° C. Charge II was added and the mixture was cooled to 70° C. Charge III was added over a 10 minute period. Charge IV was added and the resulting mixture was gradually heated to 90° C. over 90 minutes and then held at 90° C. for 1 hour. Charge V was stirred in a separate flask and heated to 60° C. 1387.8 g of the reaction product of Charges I, II, III, and IV was added to Charge V over 10 minutes. Charge VI was added and the resulting mixture was cooled to room temperature. The final product was a translucent emulsion with an acid value of 12.5, a Brookfield viscosity of 3710 centipoise (spindle #5 at 60 rpm), a pH of 7.6, and a nonvolatile content of 29.4% as measured at 110° C. for one hour.

### EXAMPLE 6

### Polyurethane/Nanopigment Dispersion

This example describes the preparation of a nano-sized PB 15:3 phthalocyanine blue pigment dispersion. The dispersion was prepared from the following mixture of ingredients in the ratios indicated:

| Ingredients | Weight (grams) |
|---|---|
| Charge I | |
| Polyurethane dispersion of Example 5 | 7271.0 |
| Deionized water | 3293.1 |
| Hydroquinone methyl ether (MEHQ) | 2.0 |
| PB 15:3 pigment | 1079.5 |
| Shellsol OMS (Shell Chemical Co.) | 131.5 |
| Charge II | |
| Deionized water | 102.4 |
| t-Butyl hydroperoxide (70% aqueous solution) | 12.3 |
| Charge III | |
| Deionized water | 512.1 |
| Ferrous ammonium sulfate | 0.15 |
| Sodium metabisulfite | 12.3 |

The ingredients of Charge I were mixed using a Ross rotor/stator mixer Model #HSM-100L for 2.5 hours and then recycled through an Advantis V15 Drais mill containing 500 ml of 0.3 mm Zirconox YTZ® grinding media in a one liter grinding chamber. The mixture was milled at 1400 rpm for a total time of 19.0 hours. The progress of the milling was monitored by visually observing changes in the transparency of thin films of samples drawn down over black and white Leneta paper. Charge II was added and the resulting mixture was stirred 5 minutes at 11° C. Charge III was added in two aliquots over 5 minutes. The temperature of the mixture increased to 13° C. The final product was a blue liquid with a Brookfield viscosity of 26 centipoise (spindle #1 at 60 rpm), a pH of 7.2, and a nonvolatile content of 30.0% as measured at 110° C. for one hour.

### EXAMPLE 7

### Polyurethane/Nanopigment Dispersion

This example describes the preparation of a nano-sized PR 122 quinacridone magenta pigment dispersion. The dispersion was prepared from the following mixture of ingredients in the ratios indicated:

| Ingredients | Weight (grams) |
|---|---|
| Charge I | |
| Polyurethane dispersion of Example 5 | 7271.0 |
| Deionized water | 3293.1 |
| Hydroquinone methyl ether (MEHQ) | 2.0 |
| PR 122 pigment | 1079.5 |
| Shellsol OMS (Shell Chemical Co.) | 131.5 |
| Charge II | |
| Deionized water | 102.4 |
| t-Butyl hydroperoxide (70% aqueous solution) | 12.3 |
| Charge III | |
| Deionized water | 512.1 |
| Ferrous ammonium sulfate | 0.15 |
| Sodium metabisulfite | 12.3 |

The ingredients of Charge I were mixed using a Ross rotor/stator mixer Model #HSM-100L for 4 hours and then recycled through an Advantis V15 Drais mill containing 500 ml of 0.3 mm Zirconox YTZ® grinding media in a one liter grinding chamber. The mixture was milled at 1400 rpm for a total time of 23 hours. The progress of the milling was monitored by visually observing changes in the transparency of thin films of samples drawn down over black and white Leneta paper. Charge II was added and the resulting mixture was stirred 5 minutes at 24° C. Charge III was added in two aliquots over 5 minutes. The temperature of the mixture increased to 26° C. The final product was a magenta liquid with a Brookfield viscosity of 27 centipoise (spindle #1 at 60 rpm), a pH of 7.4, and a nonvolatile content of 29.3% as measured at 110° C. for one hour.

### EXAMPLE 8

### Polyurethane/Nanopigment Dispersion

This example describes the preparation of a nano-sized PY 128 di-azo yellow pigment dispersion. The dispersion was prepared from the following mixture of ingredients in the ratios indicated:

| Ingredients | Weight (grams) |
|---|---|
| Charge I | |
| Polyurethane dispersion of Example 5 | 7271.0 |
| Deionized water | 3293.1 |
| Hydroquinone methyl ether (MEHQ) | 2.0 |
| PY 128 pigment | 1079.5 |
| Shellsol OMS (Shell Chemical Co.) | 131.5 |
| Charge II | |
| Deionized water | 102.4 |
| t-Butyl hydroperoxide (70% aqueous solution) | 12.3 |
| Charge III | |
| Deionized water | 512.1 |
| Ferrous ammonium sulfate | 0.15 |
| Sodium metabisulfite | 12.3 |

The ingredients of Charge I were mixed using a Ross rotor/stator mixer Model #HSM-100L for 5.5 hours and then recycled through an Advantis V15 Drais mill containing 500 ml of 0.3 mm Zirconox YTZ® grinding media in a one liter grinding chamber. The mixture was milled at 1400 rpm for a total time of 23 hours. The progress of the milling was monitored by visually observing changes in the transparency of thin films of samples drawn down over black and white Leneta paper. Charge II was added and the resulting mixture was stirred 5 minutes. Charge III was added in two aliquots over 5 minutes. The final product was a yellow liquid with a Brookfield viscosity of 53 centipoise (spindle #1 at 60 rpm), a pH of 7.3, and a nonvolatile content of 28.8% as measured at 110° C. for one hour.

### EXAMPLE 9

### Polyurethane/Pigment Dispersion

This example describes the preparation of a titanium dioxide pigment dispersion. The dispersion was prepared from the following mixture of ingredients in the ratios indicated:

| Ingredients | Weight (grams) |
|---|---|
| Charge I | |
| Polyurethane dispersion of Example 5 | 375.0 |
| Deionized water | 35.0 |
| Propylene glycol monobutyl ether | 45.0 |
| Igepal CO-897² | 4.29 |
| Tiona 595³ | 102.8 |
| Ferrous ammonium sulfate | 0.01 |
| Charge II | |
| t-Butyl hydroperoxide (70% aqueous solution) | 0.6 |
| Charge III | |
| Deionized water | 10.0 |
| Sodium metabisulfite | 0.6 |

²Ethoxylated nonylphenol available from Stepan Co.
³Titanium dioxide available from Millennium Chemicals, Inc.

The ingredients of Charge I were mixed in a water-cooled one liter jacketed stainless steel beaker with an inside diameter of 9.9 cm. The ingredients were mixed using a Premier Mill Laboratory Dispersator Model 2000 equipped with a 7.5 cm Cowles blade. The mixture was stirred at 2800 to 3100 rpm for a total time of 6 hours and then 469.4 g of the mixture was poured into a roundbottom fourneck flask. The mixture was sparged for 5 minutes with nitrogen. Charge II was added and the resulting mixture was stirred 5 minutes at 22° C. Charge III was added in two aliquots over 5 minutes. The temperature of the mixture increased to 30° C. The final product was a viscous, opaque white liquid with a pH of 6.7, and a nonvolatile content of 44.4% as measured at 110° C. for one hour.

### EXAMPLE 10

### Polyurethane/Acrylic Dispersion

This example describes the preparation of an aqueous polyurethane/acrylic dispersion. The dispersion was prepared from the following mixture of ingredients in the ratios indicated:

| Ingredients | Weight (grams) |
|---|---|
| Charge I | |
| Deionized water | 1400.0 |
| Ferrous ammonium sulfate | 0.1 |
| t-Butyl hydroperoxide (70% aqueous solution) | 5.0 |
| Ethylene glycol monobutyl ether | 500.0 |
| Polyurethane dispersion of Example 5 | 3750.0 |
| Methyl methacrylate | 750.0 |
| Butyl methacrylate | 250.0 |
| Charge II | |
| Sodium metabisulfite | 5.0 |
| Deionized water | 400.0 |

The polyurethane dispersion was prepared in a four neck round bottom flask equipped with an electronic temperature probe, mechanical stirrer, condenser, nitrogen inlet, and an addition funnel. The mixture was stirred at 26° C. under a nitrogen atmosphere. Charge II was added over 30 minutes. The temperature of the mixture increased to 58° C. The final product was a translucent liquid with a pH of 7.0, a Brookfield viscosity of 70 cps (measured with a #2 spindle at 60 rpm) and a nonvolatile content of 34.5% as measured at 110° C. for one hour.

### EXAMPLE 11

### Primary Coatings

This example describes the preparation of a set of cyan, magenta, and yellow coatings. The coatings were prepared from the following mixture of ingredients in the ratios indicated:

| | Cyan | Magenta | Yellow |
|---|---|---|---|
| Ingredients | Weight (grams) | | |
| Polyurethane/acrylic dispersion | 50.0 | 50.0 | 50.0 |
| of Example 10 | | | |
| Cyan tint of Example 6 | 38.1 | | |
| Magenta tint of Example 7 | | 39.7 | |
| Yellow tint of Example 8 | | | 39.6 |
| Dimethyl ethanolamine | 0.16 | 0.16 | 0.16 |

The coatings were prepared by stirring the ingredients in glass jars. The coatings of this example were mixed in various proportions to make fingernail coatings of various colors as described in Example 12.

### EXAMPLE 12

### Fingernail Coatings

This example describes the preparation of fingernail coatings of various colors. The coatings were prepared from the cyan, magenta, and yellow primary coatings of Example 11 and the white titanium dioxide pigment dispersion of Example 9.

Ingredient weight (grams)

| Coating color | Cyan | Magenta | Yellow | White |
|---|---|---|---|---|
| Red | | 7.0 | 3.0 | |
| Pastel red | | 1.4 | 0.6 | 0.35 |
| Pink | | 1.4 | 0.6 | 1.5 |
| Orange | | 0.5 | 9.5 | |
| Violet | 2.5 | 7.5 | | |
| Olive | 0.7 | 1.2 | 8.1 | 1.0 |

The coatings were prepared by mixing the ingredients in glass jars. The coatings were then applied with an artist's brush to CustomFit™ artificial nails (available from Kiss Products, Inc., Port Washington, N.Y.). The coatings dried at ambient temperature to form hard, glossy, intensely-colored films.

It will be appreciated by those skilled in the art that changes could be made to the embodiments described above without departing from the broad inventive concept thereof. It is understood, therefore, that this invention is not limited to the particular embodiments disclosed, but it is intended to cover modifications which are within the spirit and scope of the invention, as defined by the appended claims.

We claim:

1. A personal care composition comprising a colorant, wherein the colorant comprises a dispersion of polymer-enclosed nanoparticles.

2. The personal care composition of claim 1, wherein the colorant further comprises a radiation diffraction material comprising an ordered periodic array of particles held in a matrix.

3. The personal care composition of claim 1, wherein the composition is a cosmetic composition.

4. The cosmetic composition of claim 3, wherein the cosmetic composition is selected from the group consisting of a skin care composition, a hair care composition, a nail care composition, and a makeup composition.

5. The personal care composition of claim 1, wherein the composition is a pharmaceutical composition.

6. The personal care composition of claim 1, wherein the composition is an oral hygiene composition.

7. The personal care composition of claim 1, wherein the dispersion of polymer-enclosed nanoparticles is prepared by:
(a) a method comprising:
(1) providing a mixture, in an aqueous medium, of
(i) particles,
(ii) one or more polymerizable, ethylenically unsaturated monomers; and/or
(iii) a mixture of one or more polymerizable unsaturated monomers with one or more polymers; and/or
(iv) one or more polymers, and then
(2) subjecting the mixture to high stress shear conditions in the presence of an aqueous medium; or
(b) a method comprising:
(1) providing a mixture, in an aqueous medium, of
(i) particles,
(ii) a polymerizable ethylenically unsaturated monomer, and
(iii) a water-dispersible polymerizable dispersant, and
(2) polymerizing the ethylenically unsaturated monomer and polymerizable dispersant to form polymer-enclosed color-imparting particles comprising a water-dispersible polymer.

8. The personal care composition of claim 1, further comprising a protein.

9. The personal care composition of claim 1, further comprising a personal care active ingredient.

10. The personal care composition of claim 1, wherein the personal care active ingredient comprises an antioxidant, a vitamin or vitamin derivative, a peptide, polypeptide or a derivative thereof, an enzyme, an amino acid, a retinoid, or a mixture thereof.

11. The personal care composition of claim 9, further comprising a carrier.

12. The personal care composition of claim 1, further comprising a humectant.

13. The personal care composition of claim 1, wherein the composition is in the form of an oil-in-water emulsion or a water-in-oil emulsion.

14. The personal care composition of claim 1, further comprising an organic sunscreen, an inorganic physical sunblock, or a mixture thereof.

15. The personal care composition of claim 1, further comprising a preservative.

16. An agrochemical compositions comprising:
(a) an agricultural active ingredient; and
(b) a colorant, wherein the colorant comprises:
(i) a radiation diffraction material comprising an ordered periodic array of particles held in a matrix,
(ii) a dispersion of polymer-enclosed nanoparticles, or
(iii) a mixture thereof.

17. The agrochemical composition of claim 16, wherein the agricultural active ingredient comprises a fertilizer, a fungicide, an insecticide, a herbicide, a bactericide, or a mixture thereof.

18. The agrochemical composition of claim 17, wherein the fertilizer comprises a macronutrient, a micronutrient, or a mixture thereof.

19. The agrochemical composition of claim 16, wherein the composition is a coating composition further comprising a film-forming polymer.

20. A method for treating a keratinous substrate comprising applying to at least a portion of the substrate to be treated an effective amount of a treatment composition comprising a colorant, wherein the colorant comprises:
(i) a radiation diffraction material comprising an ordered periodic array of particles held in a matrix,
(ii) a dispersion of polymer-enclosed nanoparticles, or
(iii) a mixture thereof.
